(19) United States
(12) Patent Application Publication
(10) Pub. No.: US 2020/0222524 A1
(43) Pub. Date: Jul. 16, 2020

(54) VIRAL FORMULATIONS CONTAINING AMINO ACIDS

(71) Applicant: Vaccine Stabilization Institute, Camarillo, CA (US)

(72) Inventors: Byeong Seon Chang, Thousand Oaks, CA (US); Jeong S. Oh, Newbury Park, CA (US)

(73) Assignee: Vaccine Stabilization Institute, Camarillo, CA (US)

(21) Appl. No.: 16/828,441

(22) Filed: Mar. 24, 2020

Related U.S. Application Data
(63) Continuation of application No. PCT/US19/63898, filed on Nov. 30, 2019.
(60) Provisional application No. 62/774,047, filed on Nov. 30, 2018.

(51) Int. Cl. A61K 39/12; C12N 7/00; A61K 47/26; A61K 47/42; A61K 47/34; A61K 47/02; A61K 47/18

(52) U.S. Cl. CPC A61K 39/12; C12N 7/00; A61K 47/26; A61K 2039/525; A61K 47/34; A61K 47/02; A61K 47/183; A61K 47/42

(57) ABSTRACT

Provided are stable virus formulations that contain at least three amino acids, at least one protein, at least one carbohydrate, and at least one salt. In certain embodiments, amino acid combinations with either at least three, or four, or more amino acids are included. By virtue of inclusion of the amino acids, a virus in the formulations is physically and biologically stable both in liquid and dry powder state. In further embodiments, by virtue of inclusion of the amino acids, a virus in the formulations is physically and biologically stable during lyophilization process. In further embodiments, by virtue of inclusion of the amino acids, a virus in the formulations is physically and biologically stable during storage in both liquid and lyophilized states.

### CROSS REFERENCE TO RELATED APPLICATIONS

The present application is a national phase application of PCT/US2019/063898 and claims priority to U.S. Provisional Application No. 62/774,047 filed Nov. 30, 2018, the contents of which are incorporated by reference.

### FIELD OF INVENTION

This invention relates generally to formulations for vaccines, and more specifically, to formulations that provide stability to vaccines, viruses and viral vectors.

### BACKGROUND

The advent of vaccines has proven to be one of the greatest achievements in modern medicine. Vaccinations have been credited with the eradication of smallpox. The incidence and threat of other diseases such as polio, measles, and tetanus have been substantially reduced in much of the world.

The delivery of a virus or a preparation containing at least a portion of a virus to an animal (e.g. a patient) has many medical or therapeutic applications, for example, for vaccines, for gene therapy, for cell therapy, and for viral therapy. For these applications it is generally desirable for viral preparations and formulations to retain their activity in terms of infectivity, gene expression, or immunogenicity. While many vaccines are efficacious and can be manufactured at low cost, they can be unstable which can make them impractical or expensive for widespread use.

The efficacy or effectiveness of a vaccine depends in part on its stability during shipping and storage. Vaccines and other biologically active material can be prone to degradation, particularly when agitated or exposed to heat. Transportation and storage at ambient temperature can lead to rapid inactivation viruses. For this reason, vaccines and other biologically active products typically require low temperatures or freezing. Freeze drying (i.e. lyophilization) presents another option for some products.

Lyophilization involves drying a solution at low temperature. It can improve the stability of the dried product when stored at low temperature. However, many virus products experience significant loss of potency during the drying process. Alternatives to lyophilization generally involve adding preservatives to the vaccine solution. This can cause safety concerns. For example, thiomersal can improve the shelf-life of biologics, however it is generally avoided because of its mercury content.

Accordingly, there is a need for a solution that improves the stability of vaccines and related biologics. A solution with stability-conferring properties could lower costs and increase accessibility to vaccines, particularly in rural and under-served areas. Such a solution should improve the shelf-life of vaccines when subject to higher temperatures, agitation and freeze-thaw cycles.

There is, therefore, a need for methods to make improved viral formulations, and for the formulations themselves. In one aspect, there is a need for viral formulations that are stable during storage and transportation. In a further aspect, there is a need for formulations that are stable in liquid or lyophilized forms for administration by injection or by alternative routes of administration. Also, there is a need for better systematic methods to develop such formulations.

### SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

The experimental examples described herein demonstrate that particular amino acids can improve the stability of vaccines, viruses and viral vector in solution, without affective the viral structure.

Also described herein are processes for screening for suitable amino acid and amino acid combinations that improve the stability of viral formulations, using a high throughput screening protocol based on relevant physical and biochemical markers.

One embodiment provides a formulation for stabilizing a vaccine, virus or viral vector. The viral stabilizing solution can be referred to as “VSI” or “VSI solution.” The solution can improve the stability of a virus in solution when stored an ambient temperature or below ambient temperature. The solution can also improve the stability of a virus in solution when stored above ambient temperature, when frozen and/or when agitated. The solution can also improve the stability of a virus when lyophilized.

Thus, one embodiment provides a stabilizing formulation for a vaccine, virus or viral vector that includes three different amino acids. In some aspects, the amino acid is not Tryptophan (W), Isoleucine (I), Phenylalanine (F) or Leucine (L).

Another embodiment provides a stabilizing formulation for a vaccine, virus or viral vector that includes four different amino acids. In one aspect, the two different amino acids are not Tryptophan (W), Isoleucine (I), Phenylalanine (F) or Leucine (L).

Another embodiment provides a stabilizing formulation for a vaccine, virus or viral vector that includes five different amino acids. In one aspect, the five different amino acids are not Tryptophan (W), Isoleucine (I), Phenylalanine (F) or Leucine (L).

Another embodiment provides a stabilizing formulation for a vaccine, virus or viral vector that includes six different amino acids. In one aspect, the six different amino acids are not Tryptophan (W), Isoleucine (I), Phenylalanine (F) or Leucine (L).

Another embodiment provides a stabilizing formulation for a vaccine, virus or viral vector that includes seven different amino acids. In one aspect, the seven different amino acids are not Tryptophan (W), Isoleucine (I), Phenylalanine (F) or Leucine (L).

Another embodiment provides a stabilizing formulation for a vaccine, virus or viral vector that includes eight or more different amino acids. In one aspect, the eight or more different amino acids are not Tryptophan (W) or Phenylalanine (F).

Another embodiment provides a stabilizing formulation for a vaccine, virus or viral vector that includes three or more different amino acids, a carbohydrate, or polyhydryl alcohol or a sugar.

Another embodiment provides a stabilizing formulation for a vaccine, virus or viral vector that includes three or more different amino acids, a carbohydrate and a salt.

Another embodiment provides a stabilizing formulation for a vaccine, virus or viral vector that includes three or more different amino acids, a carbohydrate, salt and a protein.

Another embodiment provides a stabilizing formulation for a vaccine, virus or viral vector that includes three or more different amino acids and a surfactant such as F68, polysorbate 20, or polysorbate 80.

Aspects of the present invention disclose a formulation containing a mixture of amino acids to stabilize a virus in the pharmaceutical formulation. Further aspects of the invention include a formulation wherein the mixture of amino acid stabilizes the virus and carbohydrates, salt, and other protein excipients, thus enhancing the stabilizing effect.

Aspects of the invention include a stabilizing formulation, wherein the formulation includes three or more different amino acids to stabilize a virus or viral preparation. Another aspect is a stabilizing formulation, wherein the formulation comprises carbohydrates, salts, and protective proteins to reduce the inactivation at the surface and to overcome the detrimental effect of the amino acids. Thus certain embodiments are directed to formulation that includes three or more different amino acids to stabilize a virus in the formulation. Carbohydrates, a salt and a protein can reduce the surface inactivation and overcome potential detrimental effects of the amino acids.

Embodiments include a stabilizing formulation, wherein, the amino acid to stabilize the virus includes three to all fifteen amino acids selected from the following: Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), and Valine (V).

Embodiments provide a stabilizing formulation containing a mixture of amino acids and a stabilizing carbohydrate. The stabilizing carbohydrates is one or more of glucose, mannitol, sorbitol, inositol, maltose, lactose, sucrose, and trehalose.

Embodiments provide a stabilizing formulation containing a mixture of amino acids, carbohydrates and a protective protein. The protective protein is one or more of: albumin, cytokine, enzyme, immunoglobulin, antibody, or gelatin.

Embodiments provide a stabilizing formulation comprising one or more of the following viruses: Adeno-Associated Virus, Adenovirus, Arena virus (Lassa virus), Alpha virus, Astrovirus, Bacille Calmette-Guerin ‘BCG’, BK virus (including associated with kidney transplant patients), Papovavirus, Bunyavirus, Burkett's Lymphoma (Herpes), Calicivirus, California, encephalitis (Bunyavirus), Colorado tick fever (Reovirus), Corona virus, Coronavirus, Coxsackie, Coxsackie virus A, B (Enterovirus), Crimea-Congo hemorrhagic fever (Bunyavirus), Cytomegalovirus, Cytomegaly, Dengue (Flavivirus), Diptheria (bacteria), Ebola, Ebola/Marburg hemorrhagic fever (Filoviruses), Epstein-Barr Virus ‘EBV’, Echovirus, Enterovirus, Eastern equine encephalitis ‘EEE’, Togaviruses, Encephalitis, Enterovirus, Flavi virus, Hantavirus, Hepatitis A, (Enterovirus), Hepatitis B virus (Hepadnavirus), Hepatitis C (Flavivirus), Hepatitis E (Calicivirus), Herpes, Herpes Varicella-Zoster virus, HIV Human Immunodeficiency Virus (Retrovirus), HIV—AIDS (Retrovirus), Human Papilloma Virus ‘HPV’, Cervical cancer (Papovavirus), HSV 1 Herpes Simplex I, HSV 2 Herpes Simplex II, HTLV—T-cell leukemia (Retrovirus), Influenza (Orthomyxovirus), Japanese encephalitis (Flavivirus), Kaposi's Sarcoma associated herpes virus KSHV (Herpes HHV 8), Kyusaki, Lassa Virus, Lentivirus, Lymphocytic Choriomeningitis Virus LCV (Arenavirus), Measles (Rubella), Measels, Measles Micro (Paramyxovirus), Monkey Bites (Herpes strain HHV 7), Mononucleosis (Herpes), Morbilli, Mumps (Paramyxovirus), Newcastle's diseases virus, Norovirus, Norwalk virus (Calicivirus), Orthomyxoviruses (Influenza virus A, B, C), Papillomavirus (warts), Papova (M.S.), Papovavirus (JC—progressive multifocal leukoencephalopathy in HIV) (Papovavirus), Parainfluenza Nonsegmented (Paramyxovirus), Paramyxovirus, Parvovirus (B19 virusaplastic crises in sickle cell disease), Picorna virus, Pertussus (bacteria), Polio (Enterovirus), Poxvirus (Smallpox), Prions, Rabies (Rhabdovirus), Reovirus, Retrovirus, Rhabdovirus (Rabies), Rhinovirus, Roseola (Herpes HHV 6), Rotavirus, Respiratory SyncitialVirus (Paramyxovirus), Rubella (Togaviruses), Flavivirus, Poxvirus, Vaccinia virus, Variola, Venezuelan Equine Encephalitis ‘VEE’ (Togaviruses), Wart virus (Papillomavirus), Western Equine Encephalitis “WEE” (Togaviruses), West Nile Virus (Flavivirus), Yellow fever (Flavivirus) and Zika virus (ZIKV).

Aspects of the invention include a stabilizing formulation for virus, wherein the virus is adeno-associated virus (AAV), adenovirus, alpha virus, coxsakie virus, flavivirus, herpes simplex virus (HSV), lentivirus, lentiviral vector, measles, New Castle Disease virus, picorna virus, polio, poxvirus, reovirus, retrovirus, rhabdo virus, vaccinia, vesicular stomatitis virus (VSV) and zika virus (ZIKV).

The formulation can includes a mixture of three to fifteen amino acids that are selected from Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Valine (V) and Lysine (K).

One or more carbohydrates can also confer stability to the virus. The carbohydrate can be glucose, mannitol, sorbitol, inositol, maltose, lactose, sucrose, and/or trehalose. A salt can also be included such as sodium chloride, potassium chloride, magnesium chloride, manganese chloride, sodium phosphate, potassium phosphate, sodium sulfate, potassium sulfate and/or ammonium sulfate. A protective protein such as albumin, cytokine, enzyme, immunoglobulin, antibody or gelatin can also be included.

Further aspects of the invention include a stabilizing formulation, wherein three to fifteen amino acids are selected from Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Valine (V) and Lysine (K). Carbohydrates are selected from glucose, mannitol, sorbitol, inositol, lactose, maltose, sucrose, and trehalose, wherein each is present at a concentration of greater than about 0.1 mg/mL. A salt that is selected from sodium chloride, potassium chloride, magnesium chloride, manganese chloride, sodium phosphate, potassium phosphate, sodium sulfate, potassium sulfate and/or ammonium sulfate, wherein each the salt present at a concentration of greater than about 1 mM. A protective protein is selected from albumin, enzyme, cytokines, immunoglobulin, antibody, or gelatin, wherein the protein is at a concentration of greater than about 0.1 mg/mL.

In another aspect, a formulation includes at least one amino acid derivative selected from Selenocysteine, W, Y, V, Citrulline, Cystine, Gama aminobutyric acid (GABA), Ornithine, Theanine, Betaine, Carnitine, Carnosine, Creatine, Hydroxyproline, Hydroxytryptophan, N-acetyl cysteine, S-Adenosyl methionine (SAM-e), Taurine, and Tyramine.

In another aspect, a stabilizing formulation for a vaccine, virus or viral vector is provided that includes three to twenty four amino acids selected from Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), and Valine (V), Selenocysteine, Carnosine, Creatine, Hydroxyproline, Hydroxytryptophan, N-acetyl cysteine, S-Adenosyl methionine (SAM-e), Taurine, and Tyramine.

Aspects of the invention include a stabilizing formulation for a vaccine, virus or viral vector, wherein stability of a virus can be determined by techniques such as visual inspection, SDS-PAGE, IEF, size exclusion liquid chromatography (SEC-HPLC), reversed phase liquid chromatography (RP-HPLC), ion-exchange HPLC, capillary electrophoresis, light scattering, particle counting, turbidity, RFFIT, bioassays, and ELISA, plaque forming unit (PFU), PCR, flow cell cytometry, immunogenicity, genes of interested expression, and determination of expressed protein(s).

Aspects of the invention include a stabilizing formulation for a virus, wherein a virus is considered stable when the virus in formulation (a) retains its physical stability, (b) retains its chemical stability and/or (c) retains its biological stability.

Further aspects of the invention include a stabilizing formulation for a virus, wherein a virus retains its physical stability in a formulation when the virus in formulation (a) does not aggregate, (b) does not precipitate, (c) does not lose its plaque forming capability, (d) does not lose its capability to infect and (e) does not lose its capability to express specific genes in the host cells.

Additional aspects of the invention include a stabilizing formulation for a virus, wherein the physical stability of a virus in a formulation is determined by (a) visual examination of color and/or clarity, (b) UV light scattering, (c) size exclusion chromatography and/or (d) electrophoresis. Further aspects of the invention include a stabilizing formulation, wherein the chemical stability of a virus in a formulation is determined by (a) size exclusion chromatography, (b) SDS-PAGE, and/or (c) matrix assisted laser desorption ionization/time of flight mass spectrometry. Further aspects of the invention include a stabilizing formulation for a virus, wherein the biological stability of a virus in a formulation is determined by analyzing the extent that it (a) retains its plaque forming capability, (b) retains its infectivity, (c) retains its immunogenicity and/or (d) retains its capability to express its gene to specific functional protein(s).

Aspects of the invention include a stabilizing formulation for a virus, wherein the pharmaceutical formulation comprises two or more different viruses.

Aspects of the invention include a stabilizing formulation for a vaccine, virus or viral vector, wherein the stability of the vaccine, virus or viral vector can be increased in the pharmaceutical formulation. In further aspects, the formulation comprises one or more additional excipients. The one or more excipients can be buffers, tonicity modifiers, bulking agents, metal ions, chelating agents, surfactants, antioxidants, polymers, salts, proteins, and carbohydrates.

Aspects of the invention include a stabilizing formulation for a vaccine, virus or viral vector, wherein the formulation is administered to a patient orally, rectally, vaginally, parenterally, intrapulmonary, sublingually, pulmonary and or intranasal. Aspects of the present invention disclose a pharmaceutical formulation, wherein the formulation is in the form of a sold or liquid; and further aspects, wherein the formulation is in the form of a liquid, powder, tablet, a capsule, a gel tab, a lozenge, an orally dissolved strip, syrup, an oral suspension, an emulsion, a granule, a sprinkle and a pellet; and further aspects wherein the formulation is a pharmaceutical composition comprising a virus formulated in a pharmaceutical formulation.

In another aspect, a kit is provided comprising a virus formulated in a pharmaceutical formulation; and further aspects, wherein the kit comprises a package containing the pharmaceutical composition and instructions; and further aspects, wherein the kit comprises a package containing the pharmaceutical composition and a device to administer the composition to a human or animal; and further aspects, wherein the device is an injectable device; and further aspects, wherein the injectable device is selected from a syringe, pen injector, auto injector and needle free injectors; and further aspects, wherein the needle free injector is a syringe; and further aspects, wherein the syringe is prefilled with a liquid; and further aspects, wherein the syringe has a single chamber; and further aspects, wherein the syringe has dual chambers; and further aspects, wherein composition is lyophilized.

Aspects of the invention include a stabilizing formulation comprising a virus and mixture of amino acids capable of stabilizing the virus in the formulation. Certain embodiments disclose a method of preparing a pharmaceutical formulation comprising mixture of amino acids, carbohydrates, a salt, and a protective protein to stabilize a virus in the pharmaceutical formulation, wherein the mixture of amino acids with carbohydrates and a salt to stabilize a virus and the protective protein to reduce the surface inactivation or the undesirable effect of amino acids are identified through the following method: (a) pre-formulation characterization; (b) high throughput screening; and, (c) long-term stability confirmation; wherein, the amino acids, carbohydrates, a salt, and a protein identified are included in the pharmaceutical formulation to provide stability to the virus.

Aspects of the invention also include a method of stabilizing a virus in solution. The method can include using a solution with amino acids, carbohydrates, a salt, a surfactant and a protective protein to stabilize the virus.

Aspects of the invention also include a method of treating a human or animal suffering from a condition. The method can include the administration of a virus for treating the condition wherein the virus is stored in a stabilizing formulation comprising three or more amino acids to stabilize the virus.

Other features and advantages of aspects of the present invention will become apparent from the following more detailed description, taken in conjunction with the accompanying drawings, which illustrate, by way of example, the principles of aspects of the invention.

### BRIEF DESCRIPTION OF THE DRAWINGS

The accompanying drawings illustrate aspects of the present invention. In such drawings:

FIG. 1A is a graph of the percent recovery for various viruses and viral vectors stored in either the VSI formulation or a control solution.

FIG. 1B is a graph of the amount of plaque forming units detected by viruses stored in either the VSI formulation of a control solution (logarithmic scale).

FIG. 2A is a graph of the percent recovery for various viruses and viral vectors after freeze-thaw cycles in either the VSI formulation or a control solution.

FIG. 2B is a graph of the amount of plaque forming units detected by viruses stored in either the VSI formulation of a control solution (logarithmic scale).

FIG. 3 is a graph of the percent recovery of a virus stored in the VSI formulation with added surfactants: polysorbates 20 (PS-20) and 80 (PS-80) and poloxamer 188 (F-68).

FIG. 4 is a graph of the percent recovery of HSV stored at refrigerator temperature (2-8° C.) in either the VSI formulation or a control solution.

FIG. 5 is a graph of the percent recovery of adenovirus stored in the formulation (VSI) or a control solution at room temperature (25° C.).

FIG. 6A is a graph of the TCID50 of a virus (Measles vaccine (Schwartz)) in the formulation (VSI) and control solution when stored at −70° C. and 5° C.

FIG. 6B is a graph of the TCID50 of a virus (Measles vaccine (Schwartz)) in the formulation (VSI) and control solution over five weeks when stored at −70° C. and 5° C.

FIG. 7 is a graph of the percent recovery of a virus that shows the effects of amino acids (Ile, Leu, Lys, Phe, and Trp) in the presence of VSI formulation containing F-68 (VSI+F-68).

FIG. 8 is an image of a plaque forming assay showing the stability of Zika virus formulated with VSI formulation compared to control samples.

### DEFINITIONS

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human. In an embodiment, a “subject” of diagnosis or treatment is a prokaryotic or a eukaryotic cell, a tissue culture, a tissue or an animal, e.g. a mammal, including a human.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers, without limitation, to the amount of the defined component sufficient to achieve the desired chemical composition or the desired biological and/or therapeutic result. In an embodiment, that result can be the desired pH or chemical or biological characteristic, e.g., stability of the formulation.

In an embodiment, as used herein, the terms “treating,” “treatment” and the like are used herein, without limitation, to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

In an embodiment, a “pharmaceutical composition” is intended to include, without limitation, the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

As used herein, the term “comprising” is intended to mean that the compositions and methods include the listed elements, but do not exclude other unlisted elements. “Consisting essentially of” when used to define compositions and methods, excludes other elements that alters the basic nature of the composition and/or method, but does not exclude other unlisted elements. Thus, a composition consisting essentially of the elements as defined herein would not exclude trace amounts of elements, such as contaminants from any isolation and purification methods or pharmaceutically acceptable carriers, such as phosphate buffered saline, preservatives, and the like, but would exclude additional unspecified amino acids. “Consisting of” excludes more than trace elements of other ingredients and substantial method steps for administering the compositions described herein. Embodiments defined by each of these transition terms are within the scope of this disclosure and the inventions embodied therein.

As used herein, the term “amino acid” refers to either natural and/or unnatural or synthetic amino acids, including glycine and both the D and L optical isomers, amino acid analogs and peptidomimetics. In some aspects, the term amino acid refers to monomeric amino acids. Non-limiting examples of naturally existing amino acids or derivative forms that are used in various embodiments include the following (with three letter, and one letter code abbreviations included): Alanine (Ala, A); Arginine (Arg, R); Asparagine (Asn, N); Aspartic acid (Asp, D); Cysteine (Cys, C); Glutamic acid (Glu, E); Glutamine (Gln, Q); Glycine (Gly, G); Histidine (His, H); Isoleucine (Ile, I); Leucine (Leu, L); Lysine (Lys, K); Methionine (Met, M); Phenylalanine (Phe, F); Proline (Pro, P); Selenocysteine; Serine (Ser, S); Threonine (Thr, T); Tryptophan (Trp, W); Tyrosine (Tyr, Y); Valine (Val, V); Citrulline; Cystine; Gama aminobutyric acid (GABA); Ornithine; Theanine; Betaine; Carnitine; Carnosine; Creatine; Hydroxyproline; Hydroxytryptophan; N-acetyl cysteine; S-Adenosyl methionine (SAM-e); Taurine; and Tyramine.

As used herein, a “virus”' may encompass any chemical or biochemical component portion of a virus, including viral component preparations, a related particle (e.g. prion), or the like and it need not be infective or capable of self-replication. In another aspect, a virus herein may be engineered to encode a reporter gene, label, or the like to enable the monitoring and or measurement of viral viability, a viral infection, or the like. Suitable labels include, but are not limited to fluorochromes, chemiluminescent compounds, dyes, and proteins, including enzymes that allow detections and/or quantification of a virus, including those described in the Haugland, Richard P. (1996) Handbook of Fluorescent Probes and Research Chemicals (6th ed.), incorporated herein by reference.

The term “viral vector” refers to a viral genome that has been adapted into a plasmid-based technology and modified for safety through the removal of many essential genes and the separation of the viral components. The use of viral vectors is a means of gene transfer to modify a specific cell type or tissue and can be manipulated to express therapeutic genes.

Viral infectivity is defined as the number of virus particles capable to invade a host cell. This is determined by using susceptible cells to the specific virus by measuring the viral infectivity. The viral titer can be measured by such means as the TCID50 method or the plaque method, and the like, but these methods rely on the morphological change of the cell when it has been infected by the virus.

The term “vaccine” refers to a biological preparation that provides active acquired immunity to a particular disease. Some vaccines contain inactivated, but previously virulent, micro-organisms that have been destroyed with chemicals, heat, or radiation. Some vaccines contain live, attenuated microorganisms. Many of these are active viruses that have been cultivated under conditions that disable their virulent properties. Toxoid vaccines are made from inactivated toxic compounds that cause illness rather than the micro-organism. Other types of vaccines include protein subunit and conjugate vaccines.

The term “GFP expression” or “green fluorescent protein expression” refers to a reporter assay that can be used, for example, to compare and measure protein expression.

The term “PFU assay” or “plaque forming assay” refers to a method used to determine virus concentration in terms of infectious dose. The number of plaque forming units (pfu) in a virus sample reflects the quantity of virus.

The term “Tissue Culture Infectious Dose” or “TCID” refers to a procedure performed to determine the infectious titer of any virus which can cause cytopathic effects (CPE) in tissue culture over a period while cells in culture remain viable. This procedure can be performed to quantify how much infectious virus is in a preparation. The TCID50 (Median Tissue Culture Infectious Dose) is one of the methods used when verifying viral titer. TCID50 signifies the concentration at which 50% of the cells are infected when a test tube or well plate upon which cells have been cultured is inoculated with a diluted solution of viral fluid.

The term “stable formulation” such as “stable pharmaceutical formulation” as used herein in connection with the formulations described herein denotes, without limitation, a formulation, which preserves its physical stability/identity/integrity and/or chemical stability/identity/integrity and/or biological activity/identity/integrity during manufacturing, storage, transportation, and application. Various analytical techniques for evaluating virus stability are available in the art and reviewed in Felix A. Rey and Shee-Mei Lok (2018) “Common Features of Enveloped Viruses and Implications for Immunogen Design for Next-Generation Vaccines,” Cell 172, pp 1319-1334 and Guy Ungerechts et al. (2016) “Moving oncolytic viruses into the clinic: clinical-grade production, purification, and characterization of diverse oncolytic viruses,” Nature: Molecular Therapy—Methods & Clinical Development 3, 16018. Stability can be evaluated by, for example, without limitation, storage at selected climate conditions for a selected time period, by applying mechanical stress such as shaking at a selected shaking frequency for a selected time period, by irradiation with a selected light intensity for a selected period of time, or by repetitive freezing and thawing at selected temperatures. The stability may be determined by, for example, without limitation, at least one of the methods selected from the group consisting of visual inspection, SDS-PAGE, IEF, size exclusion liquid chromatography (SEC-HPLC), reversed phase liquid chromatography (RP-HPLC), ion-exchange HPLC, capillary electrophoresis, light scattering, particle counting, turbidity, RFFIT, and kappa/lambda ELISA, without limitation. For example, in the data presented in the figures, stability is assessed with reference to recovery of virus by green fluorescence protein expression capability.

All numerical designations, e.g., pH, temperature, time, concentration, and molecular weight, including ranges, are to be understood as approximations in accordance with common practice in the art. When used herein, the term “about” may connote variation (+) or (−) 1%, 5% or 10% of the stated amount, as appropriate given the context. It is to be understood, although not always explicitly stated, that the reagents described herein are merely exemplary and that equivalents of such are known in the art.

Many known and useful compounds and the like can be found in Remington's Pharmaceutical Sciences (13^{th} Ed), Mack Publishing Company, Easton, Pa.—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

As the patients and subjects of the invention method are, in addition to humans, veterinary subjects, formulations suitable for these subjects are also appropriate. Such subjects include livestock and pets as well as sports animals such as horses, greyhounds, and the like.

### DETAILED DESCRIPTION

Biological products or “biologics” can include a range of products such as vaccines, blood and blood components, allergenics, somatic cells, gene therapy, tissues, and recombinant therapeutic proteins. Biologics can be produced by methods in biotechnology and/or isolated from natural sources. In contrast to most drugs with known structures that are chemically synthesized, most biologics are complex mixtures that are not easily identified or characterized. Further, biologics tend to be unstable and sensitive to heat and agitation. Without proper storage and shipping, biologics are susceptible to inactivation and/or degradation.

Accordingly, aspects of the invention include a stabilizing formulation for a biological product such as a vaccine, virus or viral vector, wherein the formulation includes one or more amino acids in solution. The formulation can include one or more additional excipients such as buffers, tonicity modifiers, bulking agents, metal ions, chelating agents, surfactants, antioxidants, polymers, salts, proteins, and carbohydrates. The formulation can be stored and shipped as a solution or lyophilized.

In one aspect, the stabilizing formulation is a solution that includes the following components: 100 mg of the protein, 500 mg sucrose, 0.5 mg polysorbate 80, 2.2 mg monobasic sodium phosphate, monohydrate, and 6.1 mg dibasic sodium phosphate dihydrate (no preservatives), reconstituted with 10 ml sterile water. The pH of the reconstituted solution is approximately 7.2. The final concentration of the antibody is 10 mg/ml. VSI solution was tested using various vaccines, viruses and viral vectors.

In an embodiment, the formulation includes the components of Table 1:

TABLE 1

| Ingredient | Weight | |
|---|---|---|
| Protein | 100 | mg |
| Monobasic Sodium | 2.2 | mg |
| Phosphate | | |
| Dibasic Sodium Phosphate | 6.1 | mg |
| Monohydrate | | |
| Amino Acids | 1-10% | |
| Deionized Water | 10 | ml |

In an embodiment, the formulation includes the components of Table 2:

TABLE 2

| Ingredient | Weight | |
|---|---|---|
| Protein | 100 | mg |
| Sucrose | 500 | mg |
| Monobasic Sodium | 2.2 | mg |
| Phosphate | | |
| Dibasic Sodium Phosphate | 6.1 | mg |
| Monohydrate | | |
| Amino Acids | 1-10% | |
| Deionized Water | 10 | ml |

In an embodiment, the formulation includes the components of Table 3:

TABLE 3

| Ingredient | Weight | |
|---|---|---|
| Protein | 100 | mg |
| Polysorbate 80 | 0.5 | mg |
| Monobasic Sodium | 2.2 | mg |
| Phosphate | | |
| Dibasic Sodium Phosphate | 6.1 | mg |
| Monohydrate | | |
| Amino Acids | 1-10% | |
| Deionized Water | 10 | ml |

In an embodiment, the formulation includes the components of Table 4:

TABLE 4

| Ingredient | Weight | |
|---|---|---|
| Protein | 100 | mg |
| Sucrose | 500 | mg |
| Polysorbate 80 | 0.5 | mg |
| Monobasic Sodium | 2.2 | mg |
| Phosphate | | |
| Dibasic Sodium Phosphate | 6.1 | mg |
| Monohydrate | | |
| Amino Acids | 1-0% | |
| Deionized Water | 10 | ml |

In an embodiment, the formulation includes the components of Table 5:

TABLE 5

| Ingredient | | |
|---|---|---|
| Protein | 2% | |
| NaCI | 100 | mM |

| | |
|---|---|
| Sugar | 10% |
| Surfactant | 0.1% |
| Amino Acids | 1-10% |
| Virus | 1% |

The sugar can be one or more monosaccharides, disaccharides or oligosaccharides including, for example, galactose, fructose, xylose, lactose, maltose, trehalose, Sorbitol, mannitol, innositol and/or trehalose. The surfactant can be a nonionic surfactant, an ionic surfactant, a cationic surfactant or a zwitterionic surfactant including, for example, Triton X-100, polysorbate 20 (PS-20) and polysorbate 80 (PS-80), poloxamer 188 (F-68) and/or tween.

In an embodiment, the formulation includes the components of Table 6:

TABLE 6

| Ingredient | | |
|---|---|---|
| Protein | 2% | |
| NaCI | 100 | mM |

| | |
|---|---|
| Mannitol | 2% |
| Innositol | 2% |
| Trehalose | 5% |
| Poloxamer 188 (F-68) | 0.1% |
| Amino Acids | 1-5% |
| Virus | 1% |

In an embodiment, the formulation includes the components of Table 7:

TABLE 7

| Ingredient | | |
|---|---|---|
| Protein | 2% | |
| NaCI | 100 | mM |

| | |
|---|---|
| Mannitol | 2% |
| Innositol | 2% |
| Trehalose | 5% |
| Amino Acids | 5-10% |
| Virus | 1% |

In an embodiment, the formulation includes the components of Table 8:

TABLE 8

| Ingredient | | |
|---|---|---|
| Protein | 2% | |
| NaCI | 100 | mM |

| | |
|---|---|
| Mannitol | 2% |
| Innositol | 2% |
| Trehalose | 5% |
| Poloxamer 188 (F-68) | 0.1% |
| Amino Acids | 5-10% |
| Virus | 1% |

The protein can be an animal-based protein, a synthetic protein or a vegetable protein. For example, the protein can be serum albumin, recombinant proteins, cytokines, enzymes, antibodies, plasma proteins, gelatin and/or soy peptone.

In some embodiments, the water is deionized water and/or purified water. Sterile water can be preferred to prevent contamination. A particular laboratory grade of water (i.e. classified as Type I, Type II and Type III) can be used, depending on the application.

Accordingly, an aspect of the invention is to provide stabilized viral formulations comprising one or more particular amino acids to improve the stability of a virus in the formulation. In an embodiment, the invention includes the viral formulation. In a further embodiment, the invention includes the formulation of a therapeutic viral composition, preparation or the like (e.g. a vaccine) in a stable formulation. In an embodiment, the invention includes the use of three or more amino acids in a formulation to improve the stability of a viral preparation. In another embodiment, the invention includes a liquid formulation for injection into a human or animal. In an embodiment, the invention includes a solid formulation, including, a lyophilized formulation that can be reconstituted into solution prior to injection. In another embodiment, the invention includes the use of a pharmaceutical composition for therapeutic treatment.

In one embodiment, a stable virus formulation contains a virus and at least three amino acids selected based on the amino acid's ability to stabilize the virus. In one embodiment, the amino acids contain a negatively charged side chain, such as aspartic acid (D) and glutamic acid (E). In another embodiment, the amino acids contain a positively charged side chain, such as arginine (R) and lysine (K). In another embodiment, the amino acids contain a hydrophobic side chain, such as Alanine (A), methionine (M), valine (V) and Tyrosine (Y). In another embodiment, the amino acid contains a polar uncharged side chain, such as cysteine (C), histidine (H), serine (S), threonine (T), asparagine (N) and glutamine (Q). In yet another embodiment, the amino acid does not have a side chain, i.e., glycine (G) and proline (P).

In an embodiment, the amino acids are not tryptophan (W), isoleucine (I), phenylalanine (F), or leucine (L). In one embodiment, the amino acids are any three or more of alanine (A), cysteine (C), aspartic acid (D), glutamic acid (E), glycine (G), histidine (H), methionine (M), asparagine (N), proline (P), glutamine (Q), arginine (R), lysine (K), serine (S), threonine (T), tyrosine (Y), or valine (V).

As noted above, while not to be bound by a particular theory, it is contemplated that, based on the discoveries described herein, the amino acids in a virus formulation may preserve the integrity of capsid or envelope structures by protecting the virus from undesirable interactions with other molecules or surfaces. In general, the forces causing virus degradation may be thought of in terms of chemical and physical instability. Water may destabilize virus directly, or act as a solvent transferring destabilizing agents, and therefore, reducing or eliminating water may promote stabilization. Freeze- or spray-drying is based on this rational. Aqueous environments, however, may be necessary for virus structure as, for example, balanced forces of hydrophilic and hydrophobic determine desired structure in a virus.

All viruses have proteins, in particular the viral capsids and viral envelopes that are comprised of proteins and lipids. Within capsid proteins, nonpolar amino acids are typically localized to the internal core to avoid exposure to water solvent, and residues with polar side chains are typically exposed on the surface. When considering the virus as a whole, the hydrogen bonds/polarity have a cumulative effect; hydrogen bonds with the surface-accessibility of polar amino acids can be stabilizing forces and dehydration can be de-stabilizing in this regard. Lyophilization, therefore, can promote degradation and appropriate excipients can act as a stabilizing force.

In this respect, therefore, it is contemplated that a combination of polar/non-polar or hydrophilic/hydrophobic amino acid monomers serves to neutralize protein-protein, protein-lipid, capsid-surface, or envelope-surface interactions based on hydrophilic or hydrophobic profiles, which by virtue of stabilizing proteins or capsid structure within the virus also stabilizes the virus, including the retention of protein activity within each individual virus or the like, which in turn leads to the retention of biological activity.

The use of a combination of one-or-more-polar with one-or-more non-polar amino acid monomers can reduce the potential for deleterious protein-protein actions by reversibly impeding access to regions on the underlying subject biomolecule by other biomolecules in the solution.

As such, some embodiments use at least three amino acid as stabilizers permitting routine pharmaceutical processings including lyophilization while substantially limiting the deleterious effects of concomitant virus-surface interactions. Thus, one embodiment provides a stable pharmaceutical formulation comprising a virus and three amino acids. In one aspect, the amino acids are not tryptophan (W), isoleucine (I), phenylalanine (F), or leucine (L). As such, some embodiments use at least three different amino acids as stabilizers permitting routine pharmaceutical processings including lyophilization while substantially limiting the deleterious effects of concomitant virus-surface interactions. Thus, one embodiment provides a stable pharmaceutical formulation comprising a virus and four different amino acids. In one embodiment, the three different amino acids are not tryptophan (W), isoleucine (I), phenylalanine (F), or leucine (L). In one embodiment, an amino acid contains a positively charged side chain. In another aspect, an amino acid contains a negatively charged side chain. In another embodiment, an amino acid contains a hydrophobic side chain. In another aspect, an amino acid contains a polar uncharged side chain. Still in another embodiment, an amino acid contains a polar uncharged side chain and the other is selected from the group consisting of glycine (G) and proline (P). In one embodiment, at least one of the three different amino acids contains a positively charged side chain. In another embodiment, at least one of the three different amino acids contains a negatively charged side chain. In another embodiment, at least one of the three different amino acids contains a hydrophobic side chain. In another embodiment, at least one of the three different amino acids contains a polar uncharged side chain. In one embodiment, each of three different amino acids is selected from alanine (A), cysteine (C), aspartic acid (D), glutamic acid (E), glycine (G), histidine (H), methionine (M), asparagine (N), proline (P), glutamine (Q), arginine (R), lysine (K), serine (S), threonine (T), tyrosine (Y) and valine (V).

Another embodiment provides a stable virus formulation that contains at least four different amino acids. Yet another embodiment provides a stable protein formulation that contains at least five different amino acids. Still another embodiment provides a stable protein formulation that contains at least six, or seven, or eight, or nine, ten, eleven, twelve, thirteen, fourteen, or fifteen different amino acids.

In an embodiment each of the amino acids are selected from the 15 natural amino acids. In an embodiment the amino acids include natural amino acids. In an embodiment the amino acids are or include other amino acids that do not occur naturally. In embodiments the amino acids are or include synthetic amino acids that do not occur naturally.

In various embodiments a formulation is provided containing 1, 2, 3, 4, 5 or 6 or more different amino acids, as well as, for one or more viruses combined with one or more excipients. In an embodiment, the one or more different amino acids are selected based on their ability to stabilize a virus in a formulation. In another embodiment, the one or more different amino acids are formulated with additional carbohydrates. In another embodiment, the one or more different amino acids are formulated with additional salt. In another embodiment, the one or more different amino acids are formulated with additional protein. In another embodiment, the one or more different amino acids are formulated with additional carbohydrates, salt, and protein.

In some aspects, the compositions and methods described herein address the problem of providing stable virus products, while maintaining administration practicability and stability. It has now been found that amino acids and their combinations, when included in a virus formulation as described herein, permit the preparation of stable liquid formulations and lyophilized formulations. Thus, one embodiment provides a stable pharmaceutical formulation comprising a virus and three or more amino acids. In one aspect, each amino acid is present at a concentration of at least about 0.1% (w/v), or alternatively at least about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%,6.25%, 6.5%, 6.75%, 7%, 7.25%, 7.5%, 7.75%, 8%, 8.25%, 8.5%, 8.75%, 9%, 9.25%, 9.5%, 9.75%, 10%, or more (w/v).

In a further aspect, each amino acid is present at a concentration of about 0.1% (w/v) to about 10%, or alternatively at about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 25%, 3% to about 25%, 3.25% to about 10%, 3.5% to about 10%, 3.75% to about 10%, 4% to about 10%, 4.25% to about 10%, 4.5% to about 10%, 4.75% to about 10%, 5% to about 10%, (w/v).

In an embodiment, a formulation is considered stable when the virus retains some or all of its biological activity of infecting cells and expressing green fluorescence protein.

In an embodiment, a virus can be said to “retain its biological stability” in a formulation if, for example, it shows no recovery loss in plaque forming capability and/or expression of green fluorescence protein in the host cells.

In an embodiment, a virus can be said to “retain its biological activity” in a pharmaceutical formulation, if, for example, the biological activity of the virus, such as HSV, at a given time is between about 50% and about 100%, or alternatively between about 60% and about 100%, or alternatively between about 70% and about 100%, or alternatively between about 80% and about 100%, or alternatively between about 90% and about 100%, of the biological activity exhibited at the time the formulation was prepared as determined, e.g., in a plaque forming unit or expression of its gene at the host cells. In a further embodiment, a virus can be said to “retain its biological activity” in a pharmaceutical formulation, if, for example, the biological activity of the virus, such as HSV, at a given time is at least 50%, 55%, 60%, 65%, 70%, 75%, 80%, 85%, 90%, 95% or 100%.

In any of the above embodiments of the formulations containing combinations of different amino acids, the formulations can have a pH that is from about 4 to about 6 or from about 5 to about 7.5. Alternatively, in a further embodiment, the pH is from about 5 to about 6, from about 6 to about 7, or from about 6.5 to about 7.5. In an embodiment, the pH is at least about 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12. In a further embodiment, the pH of the formulation is in the range of about 2 to about 12, about 3 to about 11, about 4 to about 10, about 5 to about 9, about 6 to about 8, about 6 to about 7, about 6 to about 9, about 6 to about 10, about 5 to about 6, about 5 to about 7, about 5 to about 8, about 4 to about 9, about 4 to about 8, about 4 to about 7, about 4 to about 6, about 4 to about 5, about 3 to about 10, about 3 to about 9, about 3 to about 8, about 3 to about 7, about 3 to about 6, about 3 to about 5, about 3 to about 4, about 2 to about 11, about 2 to about 10, about 2 to about 9, about 2 to about 8, about 2 to about 7, about 2 to about 6, about 2 to about 5, about 2 to about 4, about 2 to about 3, about 7 to about 8, about 7 to about 9, about 7 to about 10, about 7 to about 11, about 7 to about 12, about 8 to about 9, about 8 to about 10, about 8 to about 11, about 8 to about 12, about 9 to about 10, about 9 to about 11, about 9 to about 12, about 10 to about 11, about 10 to about 12 or about 11 to about 12.

By virtue of the stabilizing effect of the amino acids, the compositions and methods described herein permit the preparation of stable virus formulations that can be routinely processed for pharmaceutical manufacturing including pumping, filtration, ultrafiltration, diafiltration, freeze/thawing, filling, lyophilization, etc.

By virtue of the stabilizing effect of the amino acids, the compositions and methods described herein permit the preparation of stable virus formulations that can be exposed to routine pharmaceutical operation environment including, subzero temperature, refrigeration, elevated temperature, transportation, storage, ambient temperature storage, exposure to light, and administration devices and conditions.

In still certain embodiments, the formulation includes two, or three, or four or more viruses (each of which may be selected from any of the examples set forth above or may be another virus).

The amount, type, and proportion of amino acids can be determined empirically. In some embodiments, a suitable amino acid or amino acid combination may reversibly neutralize any deleterious hydrophobicity from hydrophobic residues found on the surface of the subject virus. In solution, a virus' capsid or envelope structure is dynamic, there may be at any given time particular amino acid residues exposed to the water solvent. Thus, polar amino acids, or their mimetics as described below, may be particularly useful as a type of competitive antagonist blocking such exposed hydrophobic regions from virus-surface interactions. Moreover, in embodiments that include some proportion of hydrophobic amino acid monomers, those monomers may act to interfere with non-hydrophilic regions to prevent virus-surface deleterious effects.

### EXAMPLES

The compositions and methods described herein will be further understood by reference to the following examples, which are intended to be purely exemplary. The compositions and methods described herein are not limited in scope by the exemplified embodiments, which are intended as illustrations of single aspects only. Any methods that are functionally equivalent are within the scope of the invention. Various modifications of the compositions and methods described herein in addition to those expressly described herein will become apparent to those skilled in the art from the foregoing description and accompanying figures. Such modifications fall within the scope of the invention.

### Stabilizing Effect of VSI Solution—Static and Agitation

In the following examples, unless stated otherwise, the viral stabilizing solution (herein after referred to as “VSI” or “VSI solution”) contained 20 mM histidine buffer, 100 mM NaCl, 5% trehalose, 2% mannitol, 2% innositol and 2% albumin. The solution also included 0.25% L-Alanine, 0.25% L-Arginine, 0.25% L-Asparagine, 0.25% Glutamine, 0.25% L-Glycine, 0.25% L-Histidine, 0.25% Methionine, 0.25% L-Proline, 0.25% L-Serine, 0.25% L-Threonine, 0.25% L-Valine, 0.15% L-Tyrosine, 0.25% Aspartic Acid, 0.5% Glutamic Acid.

Various vaccines, viruses and viral vectors were tested for stability when static and agitated in this solution. In this study, herpes simplex virus (HSV), adeno-associated virus (AAV) vector, Lenitiviral vector and vaccinia virus were used. Duplicate samples were suspended in either VSI solution or a control solution. The control solutions used were DMEM for HSV and Vaccinia virus, PBS containing 0.001% F-68 for AAV vector, and PBS containing 4% lactose for Lentiviral vector.

Sample were left still (i.e. static) or stressed with agitation-induced shear stress. Specifically, agitation samples were vortexed for one hour with 1,000 rpm at ambient temperature. Static samples were stored next to agitation samples at ambient temperature for one hour. Samples were then analyzed by either expression of green fluorescence protein (GFP) or plaque forming unit (PFU) and both assays.

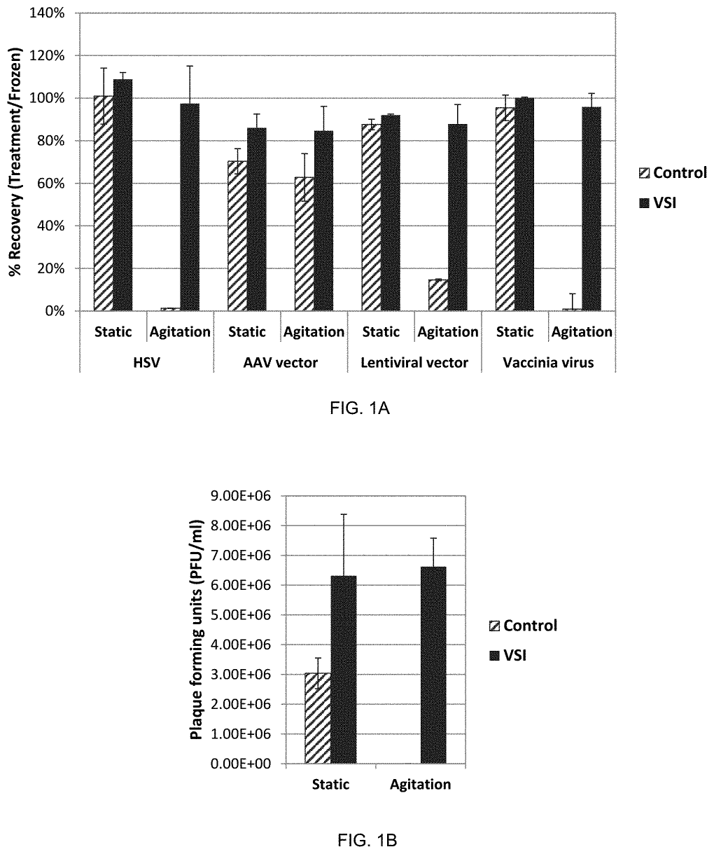

FIG. 1A shows the percent recovery for the viruses and viral vectors stored/agitated in either the VSI formulation or a control solution. The percent recovery was determined by dividing GFP expression with that of respective frozen (−70° C.) samples. The Y axis depicts the percent recovery. The samples suspended in VSI formulation are depicted with solid (filled) bars. Control samples are depicted with hashed lines.

The stability-conferring properties of the VSI solution are moderate in static samples. The stability-conferring properties of the VSI solution are more pronounced in agitated samples. The difference is most apparent in HSV and Vaccinia virus. HSV and Vaccinia virus lose nearly all of their activity with agitation. The VSI formulation prevents the loss.

FIG. 1B is a graph of the amount of plaque forming units detected by viruses stored in either the VSI formulation of a control solution (logarithmic scale). Similarly, the virus loses nearly all of its activity with agitation. The VSI formulation prevents the loss.

### Stabilizing Effect of VSI Solution—Freezing and Thawing

In this study, herpes simplex virus (HSV), adeno-associated virus (AAV) vector, Lenitiviral vector and vaccinia virus were used. As above, samples were suspended in either VSI solution or a control solution. The control solutions used were DMEM for HSV and Vaccinia virus, PBS containing 0.001% F-68 for AAV vector, and PBS containing 4% lactose for Lentiviral vector.

Sample were frozen (−70° C.) for 30 minutes and subsequently thawed at ambient temperature for 20 minutes. This free-thaw cycle was repeated for a total of three times for a first set of samples. A second set of samples underwent six freeze-thaw cycles. The percent recovery was determined by dividing GFP expression with that of respective samples maintained at 2-8° C. As above, hatched and solid bars represent control and VSI formulations, respectively.

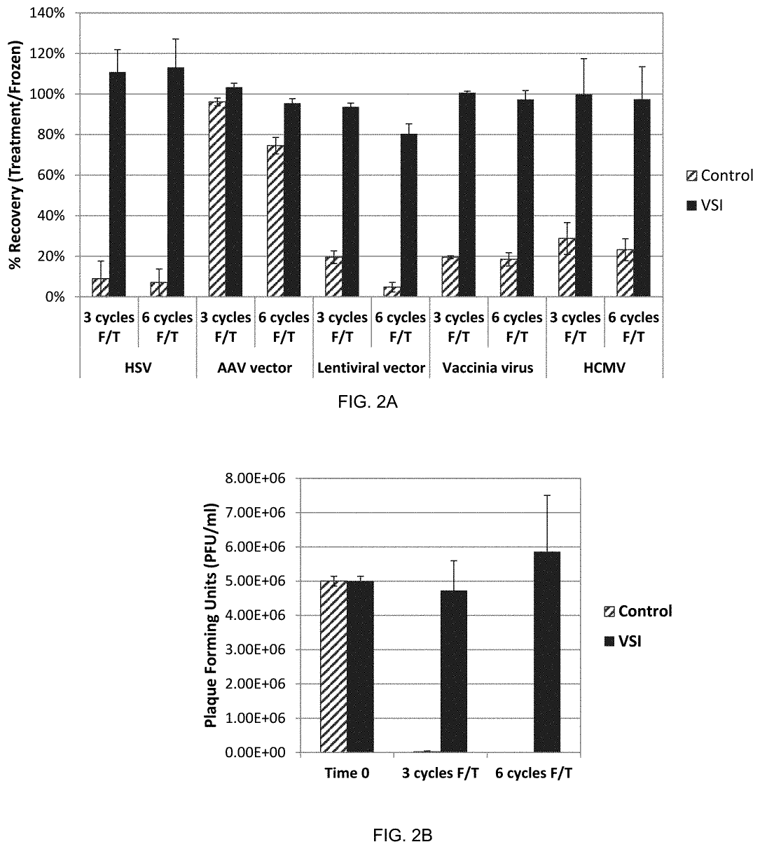

FIG. 2A shows the percent recovery for the various viruses and viral vectors stored in either the VSI formulation or a control solution. The stability-conferring properties of the VSI solution are evident in all samples. The stability-conferring properties are most pronounced in HSV, Lentiviral vector, Vaccinia virus and HCMV samples. Each control sample lost most its activity with three freeze-thaw cycles. The VSI formulation prevents the loss.

FIG. 2B shows the same samples as tested by plaque forming (PFU) assay. The Y axis represents number of PFU per milliliter. Similarly, the virus loses nearly all of its activity with three freeze-thaw cycles. The VSI formulation prevents the loss.

Stabilizing Effect of VSI with Surfactants

In this study, samples were suspended in either VSI solution or VSI solution with an added surfactant. The surfactants used were polysorbate 20 (PS-20) and polysorbate 80 (PS-80) and poloxamer 188 (F-68). Lentiviral vectors were formulated with VSI formulation with and without surfactants, treated with agitation-induced shear stress as described above and analyzed by GFP assay.

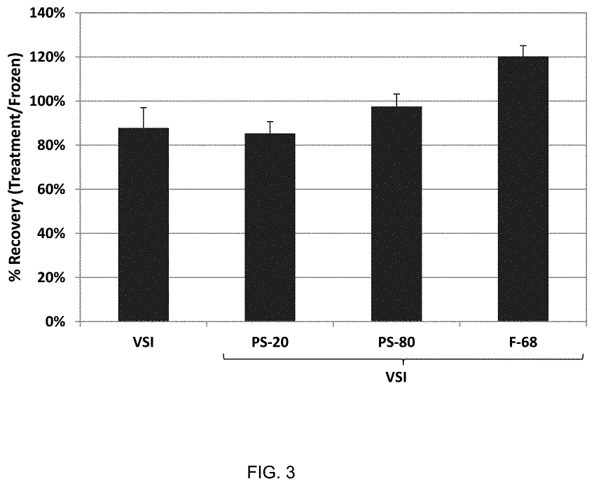

FIG. 3 shows the results; each of the surfactants added to VSI formulation further stabilized lentiviral vector against agitation-induced shear stress. The difference is most apparent with poloxamer 188 (F-68).

### Stabilizing Effect of VSI Over 30 Days

In this study, samples of HSV vector were suspended in either VSI solution or a control solution (DMEM) and stored in a refrigerator (2-8° C.). Samples were tested periodically over a 30-day period. The samples were analyzed by GFP and plaque assay. The percent recovery was determined by dividing GFP expression and plaque assay values with respective frozen (−70° C.) samples.

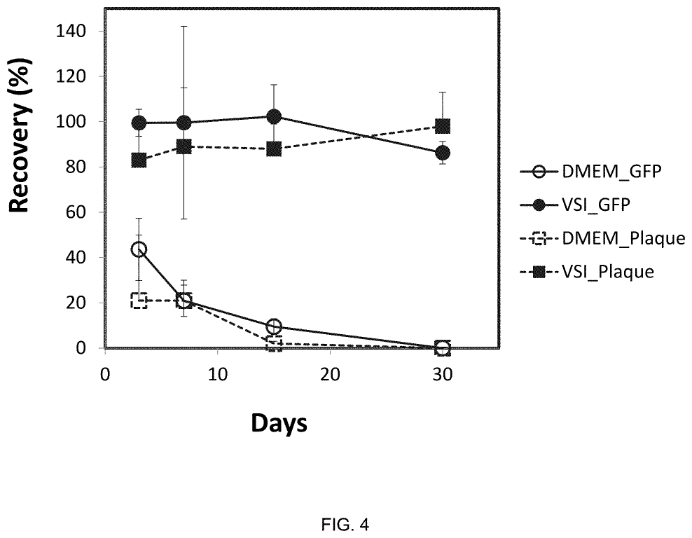

FIG. 4 shows the percent recovery of HSV during storage at refrigerated temperature (2-8° C.). Solid circles and squares represent recoveries of HSV formulated in VSI buffer by GFP and plaque assays, respectively. Open circles and squares represent recoveries of HSV formulated in DM EM buffer by GFP and plaque assays, respectively. It is noted that HSV samples lost most of their activity at 15 days. Yet, the stability-conferring properties of the VSI solution are evident throughout the 30-day period.

### Stabilizing Effect of VSI Over Eight Weeks

The study was repeated with samples of HSV formulated with either DMEM (control) or VSI buffer that were stored at room temperature (25° C.). The samples were tested periodically by GFP assay over a period of eight weeks. The percent recovery was determined by dividing GFP expression with respective frozen (−70° C.) samples.

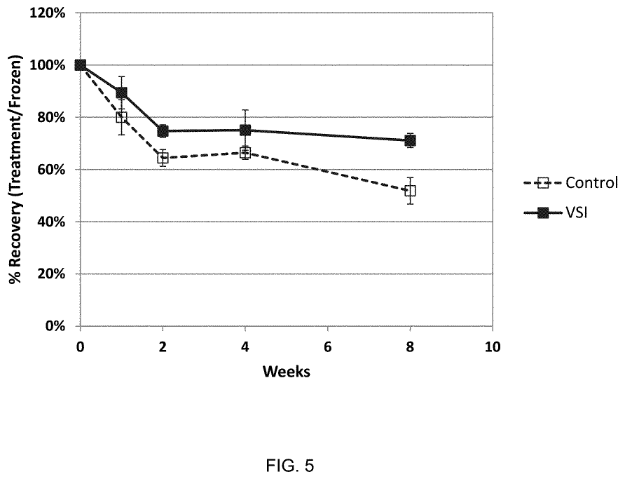

FIG. 5 shows the stability of HSV when stored at room temperature in VSI formulation and DMEM (control). Open squares represent HSV formulated in DMEM buffer. Solid squares represent HSV formulated in VSI solution. It is apparent that HSV samples stored in DMEM lost about half of their activity in eight weeks. The stability-conferring properties of the VSI solution are evident throughout the eight-week period.

### Stability of Measles Vaccine (Schwartz)

In this study, samples of measles vaccine (Schwartz) were suspended in either VSI solution or a control solution (DMEM) and stored in at either −70° C. or 5° C. Biological activities were analyzed by TCID50. TCID50 assay was performed using Vero cells expressing human SLAM gene. The 50% tissue culture infective dose (TCID50) of the Measles Schwarz vaccine were calculated using the Spearman-Kärber method as described in Hierholzer and Killington, Virology Methods Manual.

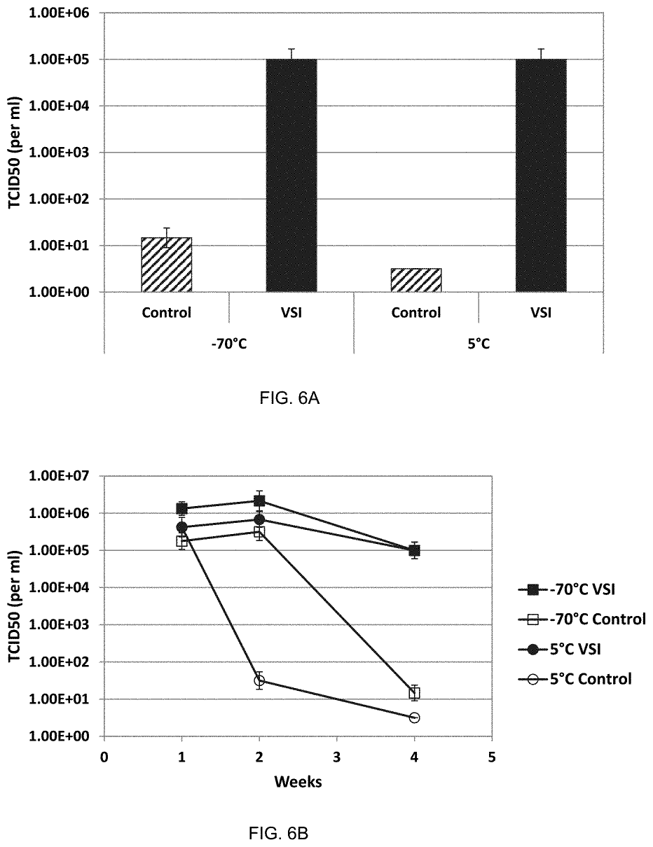

FIG. 6A shows the temperature storage data at four weeks. Hatched and solid bars represent control and VSI formulation, respectively. The stability-conferring properties of the VSI solution are evident at both −70° C. or 5° C.

FIG. 6B shows the time course of measles' biological activities when stored at −70° C. and 5° C. Solid squares and circles represent storage in VSI formulation at −70° C. and 5° C., respectively. As above, open square and circles represent storage in control formulation (DMEM) at −70° C. and 5° C., respectively.

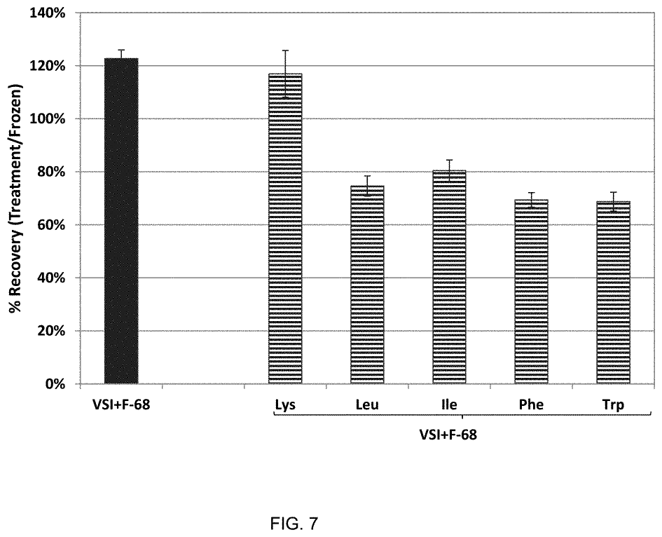

FIG. 7 shows the effects of the addition of an amino acid (Ile, Leu, Lys, Phe, and Trp). The inventors have discovered that some amino acids do not confer stability. A control solution contained VSI formulation with F-68 (VSI+F-68). Each test sample contained VSI formulation with F-68 with an additional amino acid. The percent recovery was determined by dividing GFP expression with respective frozen (−70° C.) samples.

The results demonstrate that Lysine had no significant effect on the stability-conferring properties of the VSI formulation with F-68. However, the virus was destabilized in the presence of other amino acids (i.e. Ile, Leu, Phe and Trp).

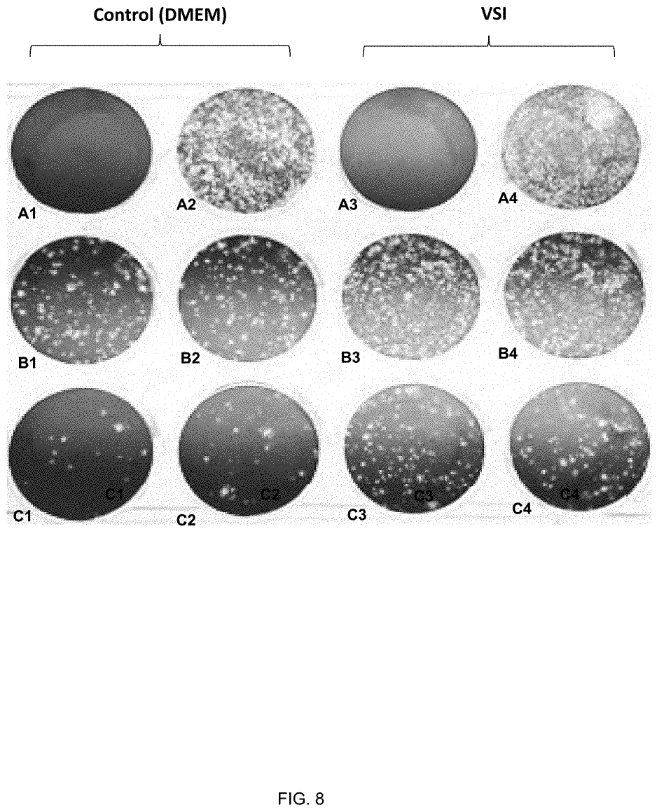

FIG. 8 shows the results of a plaque forming assay (PFU) using Zika virus. Zika virus was suspending in VSI formulation or a control solution (DMEM). Samples where then stressed with six cycles of repeated freezing and thawing and analyzed by PFU assay.

A1 and A3 wells are mock-infected wells. A2, B1, B2, C1, and C2 are wells infected with control (DMEM) formulated Zika virus. Different dilutions were used as follows:

A2: 1:1 dilution; B1 and B2: 1:10 dilution; C1 and C2: 1:100 dilution.

Similarly, A4, B3, B4, C3, and C4 wells were infected with VSI formulated Zika virus. Different dilutions were used as follows:

A4: 1:1 dilution; B3 and B4: 1:10 dilution; C3 and C4: 1:100 dilution.

The PFU results show much higher activity in wells that used VSI formulation. The number of plaques is noticeably higher in samples formulated with VSI. The difference is most apparent between B1/B2 (control) and B3/B4 (Zika). Each control sample lost activity with three freeze-thaw cycles. The VSI formulation prevented the loss. The results demonstrate greater stability of Zika virus when formulated with VSI.

### Formulation for HSV (in Solution)

HSV can be formulated in solution for parenteral administrations. In this example, the formulation contained the combination of 14 amino acids (MAA), albumin, carbohydrates, and salt. (12 amino acids: 1.25 mg/mL of Alanine (A), Arginine (R), Asparagine (N), Glutamine (Q), Glycine (G), Histidine (H), Methionine (M), Proline (P), Serine (S), Threonine (T), Tyrosine (Y), Valine (V), 2.5 mg/mL of Aspartic Acid (D), and 5 mg/mL of Glutamic Acid (E). The concentration of each carbohydrate, mannitol and inositol (MI) was 2%; the concentration of NaCl was 100 mM; the concentration of albumin was 2%. Soy peptone can be used as an alternative to albumin. The base formulation contained 20 mM histidine and HCl buffer at pH 6.0 with 5% trehalose. The formulation containing the mixture of amino acids, carbohydrate, albumin, and a salt showed much stronger expression of the green fluorescence protein than formulations without amino acids (not shown).

### Formulation for HSV (Lyophilized)

HSV can be formulated in lyophilized powder for parenteral administrations. In this example, the formulation contained the combination of 14 amino acids (MAA), albumin, carbohydrates, and salt on stability of HSV during lyophilization. (12 amino acids: 1.25 mg/mL of Alanine (A), Arginine (R), Asparagine (N), Glutamine (Q), Glycine (G), Histidine (H), Methionine (M), Proline (P), Serine (S), Threonine (T), Tyrosine (Y), Valine (V), 2.5 mg/mL of Aspartic Acid (D), and 5 mg/mL of Glutamic Acid (E). The concentration of each carbohydrate, mannitol and inositol (MI) was 2%; the concentration of NaCl was 100 mM; the concentration of albumin was 2%. The base formulation contained 20 mM histidine. HCl buffer at pH 6.0 with 5% trehalose. Individual excipients like albumin, carbohydrates, or NaCl did not sufficiently protect the virus during lyophilization. When the combination of amino acids was added to the carbohydrate, protein, and salt formulation, the virus remained stable. In addition, the formulation containing the mixture of amino acids, carbohydrate, albumin, and a salt showed much stronger expression of the green fluorescence protein than formulations without amino acids (not shown).

As provided above, in one embodiment, each amino acid can be present at a concentration of at least about 0.1% (w/v), or alternatively at least about at about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 10%, 3% to about 10%, 3.25% to about 10%, 3.5% to about 10%, 3.75% to about 10%, 4% to about 10%, 4.25% to about 10%, 4.5% to about 10%, 4.75% to about 10%, 5% to about 10%, 5.25% to about 10%, 5.5% to about 10%, 5.75% to about 10%, 6% to about 10%, 6.25% to about 10%, 6.5% to about 10%, 6.75% to about 10%, 7% to about 10%, 7.25% to about 10%, 7.5% to about 10%, 7.75% to about 10%, 8% to about 10%, 8.25% to about 10%, 8.5% to about 10%, 8.75% to about 10%, 9% to about 10%, 9.25% to about 10%, 9.5% to about 10%, 9.75% to about 10%, Still further, in an additional embodiment, each amino acid may be present at a concentration of between about 0.1 mg/ml to about 10 mg/ml, or between about 1 mg/ml to about 20 mg/ml, or between about 0.5 mg/ml to about 10 mg/ml, or between about 2 mg/ml to about 10 mg/ml. In an embodiment, each amino acid may be present at a concentration of at least about 0.01 mg/ml, 0.1 mg/ml, 0.5 mg/ml, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, or any combination thereof.

In one embodiment, any formulation as described herein is in a liquid form. In another embodiment, any formulation as described herein is provided in a container closure system that is a prefilled syringe. In an embodiment, any formulation as described herein is in a solid form. In another embodiment, any formulation as described herein is provided in a container closer system as a solid. In an additional embodiment, a solid formulation is a lyophilized solid formulation that is formed using lyophilization techniques well known in the art. In an additional embodiment, a solid formulation is a spray-dried formulation that is formed using spray-drying techniques well known in the art. In an additional embodiment, a solid formulation is a fluidized-bed powder formulation that is formed using fluidized-bed drying techniques well known in the art.

In one embodiment, a virus has, e.g., at least 50%, at least 55%, at least 60%, at least 65%, at least 70%, at least 75%, at least 80%, at least 85%, at least 90% or at least 95% of its recovery or biological activity relative to unstressed virus. In other aspects of this embodiment, a virus has, e.g., about 50% to about 100%, about 60% to about 100%, about 70% to about 100%, about 80% to about 100%, about 10% to about 90%, about 50% to about 90%, about 60% to about 90%, about 70% to about 90%, about 50% to about 80%, or about 60% to about 80%, about 50% to about 70% of its biological activity relative to native unmodified protein.

In an embodiment, a pharmaceutical formulation is comprised of a virus as an active biological agent and a soluble formulation within which the active biological agent is dissolved. In some embodiments, the soluble formulation is a formulation as described herein to further include one or more excipients, such as buffers, tonicity modifiers, bulking agents, metal ions, chelating agents, surfactants, stabilizers, polymers, salts, carbohydrates, proteins etc. As used herein, the term “excipient” refers to an inert substance which is commonly used as a diluent, vehicle, preservative, binder, or stabilizing agent for drugs and includes, but is not limited to, proteins (e.g., serum albumin, etc.), fatty acids and phospholipids (e.g., alkyl sulfonates, caprylate, etc.), surfactants (e.g., polysorbate, poloxamer, nonionic surfactant, etc.), and carbohydrates (e.g., mannitol, sorbitol, sucrose, maltose, trehalose, etc.). Also see Remington's Pharmaceutical Sciences (by Joseph P. Remington, 18th ed., Mack Publishing Co., Easton, Pa.) and Handbook of Pharmaceutical Excipients (by Raymond C. Rowe, 5th ed., APhA Publications, Washington, D.C.) which are hereby incorporated in its entirety. The excipients may impart a beneficial physical property to the formulation, such as increased product stability and increased product solubility.

In an embodiment, a composition of the invention can be free of animal products. For example, soy peptone can be used as an alternative to albumin.

In an embodiment, a composition of the invention, including, without limitation a therapeutic compound containing a virus, may include one or more carbohydrates such as a sugar, a derivatized sugar such as an alditol, aldonic acid, an esterified sugar, and/or a sugar polymer. Specific carbohydrate excipients include, for example, without limitation: monosaccharides, such as fructose, galactose, glucose, D-mannose, sorbose, and the like; disaccharides, such as lactose, sucrose, maltose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

In an embodiment, pharmaceutical compositions of the invention, including, a therapeutic compound, are potato and corn-based starches such as sodium starch glycolate and directly compressible modified starch. In an embodiment, syrups and elixirs may be formulated, without limitation, sweetening agents, for example glycerol, propylene glycol, sorbitol or sucrose. In an additional embodiment, such formulations may also contain, without limitation, a demulcent, a preservative, flavoring agents, and coloring agents.

In an embodiment, liquid suspensions can be formulated by suspending a therapeutic compound disclosed herein in a mixture with excipients suitable for the manufacture of aqueous suspensions. In an embodiment, such excipients are suspending agents, for example, sodium carboxymethylcellulose, methylcellulose, hydroxypropylmethylcellulose, sodium alginate, pectin, polyvinyl pyrrolidone, polyvinyl alcohol, natural gum, agar, gum tragacanth and gum acacia; dispersing or wetting agents may be a naturally occurring phosphatide, for example lecithin, or condensation products of an alkylene oxide with fatty acids, for example, polyoxyethylene stearate, or condensation products of ethylene oxide with long-chain aliphatic alcohols, for example, heptadecaethyleneoxycetanol, or condensation products of ethylene oxide with partial esters derived from fatty acids, for example, without limitation, polyoxyethylene sorbitan monooleate.

Particular excipients as approved for U.S. Food and Drug regulatory purposes can be found at the FDA Inactive Ingredient Database. Many useful excipients are well known in the art and can be found described in, for example, Banga, A. K., Therapeutic Peptides and Proteins, Formulation, Processing and Delivery Systems, (2d Ed 2006, CRC Press), Chapter 4, section 4.4, Pharmaceutical excipients in formulations (at pages 104-116). Any one or more of any other excipients or others may be included in any formulation as described herein. Similarly, in an embodiment, at least one excipient can confer more than one of the functions onto a formulation. Alternatively, in another embodiment, two or more excipients can be included in a formulation to perform more than one of the above or other functions. For example, an excipient can, without limitation, be included as a component in a formulation to change, adjust or optimize the osmolality of the formulation, thereby acting as a tonicity modifier.

Given the teachings and guidance provided herein, those skilled in the art will understand that a formulation described herein can be equally applicable to many types of biopharmaceuticals, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art. For example, non-reducing sugars exhibit favorable excipient properties when used with polypeptide biopharmaceuticals compared to reducing sugars. Accordingly, exemplary formulations are exemplified further herein with reference to polypeptide biopharmaceuticals. However, the range of applicability, chemical and physical properties, considerations and methodology applied to polypeptide biopharmaceutical can be similarly applicable to biopharmaceuticals other than polypeptide biopharmaceuticals.

In various embodiments, a formulation can include, without limitation, combinations of bioactive agents (such as viruses, proteins, antibodies, peptides and the like as described herein) in the formulation. For example, a formulation as described herein can include a single bioactive agent for treatment of one or more conditions, including without limitation, disease. A formulation as described herein also can include, in an embodiment, without limitation, two or more different bioactive agents for a single or multiple conditions. Use of multiple bioactive agents in a formulation can be directed to, for example, the same or different indications. Similarly, in another embodiment, multiple bioactive agents can be used in a formulation to treat, for example, both a pathological condition and one or more side effects caused by the primary treatment. In a further embodiment, multiple bioactive agents also can be included, without limitation, in a formulation as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single formulation can be sufficient for some or all suggested treatments and/or diagnosis. Those skilled in the art will know those bioactive agents that can be admixed for a wide range of combination therapies. Similarly, in various embodiments, a formulation can be used with a small molecule drug and combinations of one or more bioactive agents s together with one or more small molecule pharmaceuticals. Therefore, in various embodiments a formulation is provided containing 1, 2, 3, 4, 5 or 6 or more different bioactive agents, as well as, for one or more bioactive agents combined with one or more small molecule pharmaceuticals.

In various embodiments, a formulation can include, one or more preservatives and/or additives known in the art. Similarly, a formulation can further be formulated, without limitation, into any of various known delivery formulations. For example, in an embodiment, a formulation can include, surfactants, adjuvant, biodegradable polymers, hydrogels, etc., such optional components, their chemical and functional characteristics are known in the art. Similarly known in the art are formulations that facilitate rapid, sustained or delayed release of the bioactive agents after administration. A formulation as described can be produced to include these or other formulation components known in the art.

Once a formulation is prepared as described herein, stability of the one or more bioactive agents contained within the formulation can be assessed using methods known in the art. Several methods are exemplified herein in the Examples and include plaque forming unit, PCR, determination of expressed proteins, size exclusion chromatography, particle counting and cation exchange chromatography. Other methods can comprise any of a variety of functional assays including, for example, binding activity, other biochemical activity and/or physiological activity can be assessed at two or more different time points to determine the stability of the bioactive agents in a formulation as described herein. A formulation can, in general, be prepared according to pharmaceutical standards and using pharmaceutical grade reagents. Similarly, a formulation can be prepared using sterile reagents in a sterile manufacturing environment or sterilized following preparation. Sterile injectable solutions can be prepared using known procedures in the art including, for example, by incorporating one or more bioactive agents s in the required amount in a glutamic acid buffer or excipient with one or a combination of formulation components described herein followed by sterilization microfiltration. In various embodiments, sterile powders for the preparation of sterile injectable solutions can include, for example, vacuum drying and freeze-drying (lyophilization). Such drying methods will yield a powder of the one or more bioactive agents s together with any additional desired components from a previously sterile-filtered solution thereof.

In an embodiment, further representative excipients include, without limitation, inorganic salt or buffers such as citric acid, sodium chloride, magnesium chloride, manganese chloride, potassium chloride, sodium sulfate, ammonium sulfate, potassium nitrate, sodium phosphate monobasic, sodium phosphate dibasic, and combinations thereof.

In an embodiment, further representative excipients include, without limitation polypeptides, e.g., proteins or peptides, such as serum albumin, cytokines, immunoglobulins, enzymes, gelatin, plasma proteins, etc.

In an embodiment, formulations suitable for nasal administration, wherein the carrier is a solid, include a coarse powder having a particle size, for example, in the range of about 0.2 to about 500 microns. In a further embodiment, particle size is, at least, 0.2 microns, 0.5 microns, 1 microns, 5 microns, 10 microns, 20 microns, 30 microns, 40 microns, 50 microns, 60 microns, 70 microns, 80 microns, 90 microns, 100 microns, 110 microns, 120 microns, 130 microns, 140 microns, 150 microns, 160 microns, 170 microns, 180 microns, 190 microns, 200 microns, 210 microns, 220 microns, 230 microns, 240 microns, 250 microns, 260 microns, 270 microns, 280 microns, 290 microns, 300 microns, 310 microns, 320 microns, 330 microns, 340 microns, 350 microns, 360 microns, 370 microns, 380 microns, 390 microns, 400 microns, 410 microns, 420 microns, 430 microns, 440 microns, 450 microns, 460 microns, 470 microns, 480 microns, 490 microns, 500 microns, 600 microns, 700 microns, 800 microns, 900 microns, 1000 microns. In an additional embodiment, such a formulation is typically administered, without limitation, by rapid inhalation through the nasal passage, for example, from a container of the powder held in proximity to the nose. In an additional embodiment, such a formulation is typically administered, by rapid inhalation through the mouth, for example, from a container of the powder held in proximity to the mouth. In an embodiment, a formulation for nasal delivery can be in the form of a liquid, e.g., a nasal spray or nasal drops.

In an embodiment, aerosolizable formulations for inhalation can be in dry powder form (e.g., suitable for administration by a dry powder inhaler), or, alternatively, may be in liquid form, e.g., for use in a nebulizer. In an embodiment, nebulizers for delivering an aerosolized solution include the AERx™ (Aradigm), the Ultravent® (Mallinkrodt), and the Acorn II® (Marquest Medical Products). In an embodiment, a composition of the invention can also be delivered using a pressurized, metered dose inhaler (MDI), e.g., the Ventolin® metered dose inhaler, containing a solution or suspension of a combination of drugs as described herein in a pharmaceutically inert liquid propellant, for example, a chlorofluorocarbon or fluorocarbon.

In an embodiment, formulations suitable for parenteral administration include aqueous and non-aqueous isotonic sterile solutions suitable for injection, as well as aqueous and non-aqueous sterile suspensions. In an embodiment, parenteral formulations of the invention are optionally contained in unit-dose or multi-dose sealed containers, for example, ampoules and vials, and can be stored in a freeze-dried (lyophilized) condition requiring only the addition of the sterile liquid carrier, for example, water for injections, immediately prior to use.

The composition can therefore be administered as a single dose, or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the bioactive agents in formulations described herein can, without limitation, be administered to patients throughout an extended time period, such as chronic administration for a chronic condition. The composition can be a solid, a semi-solid or an aerosol and a therapeutic compound is formulated as a tablet, geltab, lozenge, orally dissolved strip, capsule, syrup, oral suspension, emulsion, granule, sprinkle or pellet.

In an embodiment, a drug delivery platform includes both a sustained release drug delivery platform and an extended release drug delivery platform. In an embodiment, the term “sustained release” refers to the release of a therapeutic compound or compounds disclosed herein over a period of about seven days or more. In an embodiment, the term “extended release” refers to the release of a therapeutic compound or compounds disclosed herein over a period of time of less than about seven days. In an embodiment, a sustained release drug delivery platform releases a therapeutic compound or compounds disclosed herein with substantially zero order release kinetics over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In another embodiment, a sustained release drug delivery platform releases a therapeutic compound disclosed herein with substantially zero order release kinetics over a period , without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

In an embodiment, for oral, rectal, vaginal, parenteral, pulmonary, sublingual and/or intranasal delivery formulations, tablets can be made by compression or molding, optionally with one or more accessory ingredients or additives. In an embodiment, compressed tablets are prepared, for example, by compressing in a suitable tabletting machine, the active ingredients in a free-flowing form such as a powder or granules, optionally mixed with a binder (for example, without limitation, povidone, gelatin, hydroxypropylmethyl cellulose), lubricant, inert diluent, preservative, disintegrant (for example, without limitation, sodium starch glycolate, cross-linked povidone, cross-linked sodium carboxymethyl cellulose) and/or surface-active or dispersing agent.

In an embodiment, molded tablets are made, for example, without limitation, by molding in a suitable tabletting machine, a mixture of powdered compounds moistened with an inert liquid diluent. In an embodiment, the tablets may optionally be coated or scored, and may be formulated so as to provide slow or controlled release of the active ingredients, using, for example, without limitation, hydroxypropylmethyl cellulose in varying proportions to provide the desired release profile. In an embodiment, tablets may optionally be provided with a coating, without limitation, such as a thin film, sugar coating, or an enteric coating to provide release in parts of the gut other than the stomach. In an embodiment, processes, equipment, and toll manufacturers for tablet and capsule making are well-known in the art.

In an embodiment, capsule formulations can utilize either hard or soft capsules, including, without limitation, gelatin capsules or vegetarian capsules such as those made out of hydroxymethylpropylcellulose (HMPC). In an embodiment, a type of capsule is a gelatin capsule. In an embodiment, capsules may be filled using a capsule filling machine such as, without limitation, those available from commercial suppliers such as Miranda International or employing capsule manufacturing techniques well-known in the industry, as described in detail in Pharmaceutical Capules, 2.sup.nd Ed., F. Podczeck and B. Jones, 2004. In an embodiment, capsule formulations may be prepared, without limitation, using a toll manufacturing center such as the Chao Center for Industrial Pharmacy & Contract Manufacturing, located at Purdue Research Park.

The formulation described in this specification may also comprise more than one therapeutic virus as desired for the particular indication being treated, preferably those with complementary activities that do not adversely affect the other viruses. The formulations to be used for in vivo administration can be sterile. This can be accomplished, for instance, without limitation, by filtration through sterile filtration membranes, prior to, or following, preparation of the formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the LyoTip which is a prefilled syringe designed to conveniently deliver lyophilized formulations available from LyoTip, Inc., Camarillo, Calif., U.S.A. Administration by injection may be, without limitation intravenous, intramuscular, intraperitoneal, or subcutaneous, as appropriate. Administrations by non-injection route may be, without limitation, nasal, oral, cocular, dermal, or pulmonary, as appropriate.

In certain embodiments, kits can comprise, without limitation, one or more single or multi-chambered syringes (e.g., liquid syringes and lyosyringes) for administering one or more formulations described herein. In various embodiments, the kit can comprise formulation components for parenteral, subcutaneous, intramuscular or IV administration, sealed in a vial under partial vacuum in a form ready for loading into a syringe and administration to a subject. In this regard, the composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide compositions in accordance therewith. In various embodiment the kits can contain a lyophilate and a sterile diluent for reconstituting the lyophilate.

Imaging components can optionally be included and the packaging also can include written or web-accessible instructions for using the formulation. A container can include, for example, a vial, bottle, syringe, pre-filled syringe or any of a variety of formats well known in the art for multi-dispenser packaging.

Also described herein, are methods for treating a subject in need of therapy, comprising administering to the subject an effective amount of a formulation as described herein. The therapeutically effective amount or dose of a formulation will depend on the disease or condition of the subject and actual clinical setting.

In an embodiment, a formulation as described herein can be administered by any suitable route, specifically by parental (including subcutaneous, intramuscular, intravenous and intradermal) administration. It will also be appreciated that the preferred route will vary with the condition and age of the recipient, and the disease being treated. Methods of determining the most effective means and dosage of administration are known to those of skill in the art and will vary, without limitation, with the composition used for therapy, the purpose of the therapy, and the subject being treated. Single or multiple administrations can be carried out, without limitation, the dose level and pattern being selected by the treating physician. Suitable dosage formulations and methods of administering the agents are known in the art.

The formulations as described herein can be used in the manufacture of medicaments and for the treatment of humans and other animals by administration in accordance with conventional procedures.

Also provided herein are combinatorial methods for developing suitable virus formulations using combinations of amino acids. These methods are effective for developing stable liquid or lyophilized formulations, and particularly pharmaceutical virus formulations.

The general process for developing a formulation is discussed below. The process can be divided into three parts: Preformulation Characterization, High Throughput Screening and Long-Term Stability Confirmation.

Preformulation characterization studies generally are designed to understand pharmaceutically significant physicochemical properties of the formulant, such as stability when exposed to common stresses, developing assays for degradation products and other measures of stability, determining if a lyophilized or liquid formulation will be better for initial clinical studies, and developing a final research protocol. Preformulation characterization generally involves physiochemical characterization, stability assay development and stress studies to identify formulation and stability problems and facilitate optimization studies.

High throughput screening typically is used to test a large number of possible formulations to identify a limited number of candidates for development. Most degradation reactions of viruses to evaluate stability, e.g., recovery loss, can be accelerated by lyophilization and/or by increasing the storage temperature. A high throughput screening protocol can be developed based on critical issues observed at the preformulation characterization. Forced degradation may be used to expedite the relevant degradation pathway(s). Effective stabilizers or their combinations, e.g., amino acids, are identified by the high throughput screening.

A small number of promising formulations may be selected and tested for stability during lyophilization as well as long term stability. For example, samples formulated with the selected stabilizer(s) can be prepared in appropriate container/closure system, lyophilized and incubated at appropriate storage conditions or expose other relevant stresses, and characterized at appropriate time points which can last few years. Various analytical methods are used to evaluate the integrity of the virus during lyophilization followed by storage and/or or other relevant stresses. The stabilizers effective under forced degradation studies should be effective in stabilizing the protein under real life handling, storage, and transportation conditions.

Methods herein described can be useful for carrying out any one or all three stages of the development process, but, are particularly useful for systematically screening the combinatorial amino acid space for those amino acid-containing formulations most likely to provide the desired formulation characteristics and long term stability.

An embodiment provides methods for developing virus formulations with desirable properties, such stability, improved recovery, less aggregate, etc. Determining the stability of viruses has been challenging due to the instability of viruses during analyses and also inherent variability of biological assays. Considering that various reactions can compromise the viruses, e.g., loss of capsid or envelope structures, loss of capability to infect host cells, structural changes of viral genetic material, loss of capability to express specific genes into proteins in host cells, stability indicating assays for accurately assessing stability has been limited. Few stability indicating assays currently available for high throughput screening include biological assays like RFFIT, ELISA, plaque forming unit (PFU), PCR, flow cytometry, immunogenicity, and determination of expressed protein(s) and other secondary biochemical markers like SDS-PAGE, IEF, size exclusion liquid chromatography (SEC-HPLC), reversed phase liquid chromatography (RP-HPLC), ion-exchange HPLC, capillary electrophoresis, light scattering, particle counting, and turbidity.

In an embodiment in this regard, test compositions are formulated comprising a virus with mixture of amino acids and the stability and/or recovery is determined. If desired recovery is achieved, then selection of key amino acids is evaluated for further development.

Optionally, each of the selected compositions is formulated with the virus and the mixture of all amino acids without one specific amino acid. These formulations are tested stability and/or recovery. If the stability or recovery is compromised without the specific amino acid, the amino acid is selected for further development. Optionally, each of the selected compositions is formulated with the virus and the mixture of all amino acids without two specific amino acids. These formulations are tested stability and/or recovery. If the stability or recovery is compromised without the specific amino acids, the amino acids are selected for further development.

Optionally, each of the selected compositions is formulated with the virus and the mixture of all amino acids without three specific amino acids. These formulations are tested stability and/or recovery. If the stability or recovery is compromised without the specific amino acids, the amino acids are selected for further development.

Optionally, each of the selected compositions is formulated with the virus and the mixture of all amino acids without four specific amino acids. These formulations are tested stability and/or recovery. If the stability or recovery is compromised without the specific amino acids, the amino acids are selected for further development.

Optionally, each of the selected compositions is formulated with the virus and the mixture of all amino acids without five specific amino acids. These formulations are tested stability and/or recovery. If the stability or recovery is compromised without the specific amino acids, the amino acids are selected for further development.

Optionally, each of the selected compositions is formulated with the virus and the mixture of all amino acids without six specific amino acids. These formulations are tested stability and/or recovery. If the stability or recovery is compromised without the specific amino acids, the amino acids are selected for further development.

Optionally, each of the selected compositions is formulated with the virus and the mixture of all amino acids without seven specific amino acids. These formulations are tested stability and/or recovery. If the stability or recovery is compromised without the specific amino acids, the amino acids are selected for further development.

Optionally, the procedure can be repeated with additional amino acids, amino acid derivative and other substances.

Compositions in accordance with embodiments described herein have desirable properties, such as desirable solubility, viscosity, syringeabilty and stability. Lyophilates in accordance with embodiments described herein have desirable properties, as well, such as desirable recovery, stability and reconstitution.

Aspects of the present specification may also be described as follows: a formulation that includes three or more amino acids, carbohydrates, a protein, and a salt to stabilize a virus in the pharmaceutical formulation. The amino acids can stabilize the virus both in liquid and lyophilized state.

In an embodiment, the formulation includes a virus, one or more amino acids at a concentration of about 1-10% w/w, a salt at about 0.01% to 5% w/w, a carbohydrate at about 0.01% to 10% w/w, a protein at about 0.01% to 4% w/w, and water. The one or more amino acids can be at least three of Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Lysine (K) and/or Valine (V). Embodiments also include a method of stabilizing a virus using the formulation.

In an embodiment, the formulation includes four or more different amino acids to stabilize virus. The amino acids can be one or more of (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

In an embodiment, the formulation includes five or more different amino acids to stabilize virus. The amino acids can be one or more of (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V);

In an embodiment, the formulation includes six or more different amino acids to stabilize virus. The amino acids can be one or more of (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

The formulation can include a surfactant to improve stability of the virus.

The surfactant can be, for example, polysorbate 20 (PS-20), polysorbate 80 (PS-80) or poloxamer 188 (F-68).

In an embodiment, the protein is one or more of serum albumin, recombinant proteins, cytokines, enzymes, antibodies, plasma proteins, gelatin and soy peptone. Further, the protein can be an enzyme, a cytokine, a neurotropic factor, an antibody, a peptide, a hormone, a plasma protein like albumin. The protein can be present at a concentration of greater than about 10 μg/mL

In an embodiment, at least one of the amino acids contains a negatively charged side chain.

In an embodiment, at least one of the amino acids contains a negatively charged side chain.

In an embodiment, at least one of the amino acids contains a positively charged side chain

In an embodiment, at least one of the amino acids contains a hydrophobic side chain.

In an embodiment, at least one of the amino acids contains a polar uncharged side chain.

The formulation can have three different amino acids. In an embodiment, the two different amino acids have a polar uncharged side chain and the one of the two different amino acids has a hydrophobic side chain.

In an embodiment, one of the two different amino acids contains a polar uncharged side chain and the other is selected from the group consisting of D and E.

In an embodiment, the amino acids are selected from of A, R, N, D, E, Q, G, H, M, P, S, T, Y, K and V. The amino acid concentration can be greater than about 0.1 mg/mL.

The formulation can have a carbohydrate. The carbohydrates can be glucose, fructose, lactose, maltose, mannitol, sorbitol, inositol, sucrose, trehalose. One or more can be present present at a concentration of greater than about 0.1 mg/m.

The formulation can have a salt. The salt can be selected from sodium chloride, potassium chloride, magnesium chloride, manganese chloride, sodium phosphate, potassium phosphate, sodium sulfate, potassium sulfate, ammonium sulfate. The salt can have a concentration of greater than about 10 mM.

Embodiments also include a stable pharmaceutical formulation for stabilizing a HSV virus that includes at least three amino acids. At least one amino acid can be glutamic acid (E) or aspartic acid (D); and at least one amino acid can be Alanine (A), Arginine (R), Asparagine (N), Glutamine (Q), Glycine (G), Histidine (H), Methionine (M), Proline (P), Serine (S), Threonine (T), Tyrosine (Y), Lysine (K) and Valine (V).

Embodiments also include a formulation for stabilizing an enveloped virus that includes a surfactant and at least three amino acids. At least one amino acid can be glutamic acid (E) or aspartic acid (D); at least one amino acid can be Alanine (A), Arginine (R), Asparagine (N), Glutamine (Q), Glycine (G), Histidine (H), Methionine (M), Proline (P), Serine (S), Threonine (T), Tyrosine (Y), Lysine (K) or Valine (V). The enveloped virus can be, for example, herpes simplex virus (HSV), lentivirus or lentiviral vector.

Embodiments also include a formulation for stabilizing a HSV virus that includes at least three amino acids. The amino acids can be glutamic acid (E) and aspartic acid (D), and an amino acid selected from Alanine (A), Arginine (R), Asparagine (N), Glutamine (Q), Glycine (G), Histidine (H), Methionine (M), Proline (P), Serine (S), Threonine (T), Tyrosine (Y), Lysine (K) and Valine (V).

Embodiments also include a formulation for stabilizing a HSV virus that includes at least three amino acids. At least one amino acid can be selected from glutamine (G), glutamic acid (E), histidine (H), Alanine (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), and Valine (V).

Another amino acid can be an amino acid analog selected from Selenocysteine, Citrulline, Cystine, Gama aminobutyric acid (GABA), Ornithine, Theanine, Betaine, Carnitine, Carnosine, Creatine, Hydroxyproline, Hydroxytryptophan, N-acetyl cysteine, S-Adenosyl methionine (SAM-e), Taurine, and Tyramine.

In another embodiment, the formulation includes a virus, one or more amino acids at a concentration of about 1-10% w/w, a salt at about 0.01% to 5% w/w, a carbohydrate at about 0.01% to 10% w/w, a surfactant at about 0.01% to 10% w/w, and water. The pH can be from about 3 to 9. The viral formulation can preserve virus activity. Embodiments also include a method of stabilizing a virus using the formulation. The surfactant can be polysorbate 20 (PS-20), polysorbate 80 (PS-80) or poloxamer 188 (F-68). The formulation can also include a protein at about 0.01% to 4% w/w.

At least one of the one or more amino acids can have a negatively charged side chain. At least one of the one or more amino acids can have a positively charged side chain. At least one of the one or more amino acids can have a hydrophobic side chain. At least one of the one or more amino acids can have a polar uncharged side chain. Alternatively, a first amino acid can have a polar uncharged side chain and a second amino acid can have a hydrophobic side chain. Each of the amino acids can have a concentration of 0.05% to 0.5%. Embodiments also include a method of stabilizing a virus using the formulation.

In an embodiment, the amino acids can be selected from three of more of Alanine (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

Embodiments also include a pharmaceutical formulation to stabilize a HSV virus. The formulation can include at least three amino acids selected from Alanine (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

Embodiments also include a pharmaceutical formulation to stabilize a HSV virus. The formulation can include at least four amino acids selected from Alanine (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

Embodiments also include a pharmaceutical formulation to stabilize a HSV virus. The formulation can include at least five amino acids selected from Alanine (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

Embodiments also include a pharmaceutical formulation to stabilize a HSV virus. The formulation can include at least six amino acids selected from Alanine (A), Arginine (R), Asparagine (N), Aspartic Acid (D), Glutamic Acid (E), Glutamine (Q), Glycine (G), Histidine (H), Methionine (M), Proline (P), Serine (S), Threonine (T), Tyrosine (Y), Lysine (K) and Valine (V).

The virus can be selected from Adeno-Associated Virus, Adenovirus, Arena virus (Lassa virus), Alpha virus, Astrovirus, Bacille Calmette-Guerin ‘BCG’, BK virus (including associated with kidney transplant patients), Papovavirus, Bunyavirus, Burkett's Lymphoma (Herpes), Calicivirus, California, encephalitis (Bunyavirus), Colorado tick fever (Reovirus), Corona virus, Coronavirus, Coxsackie, Coxsackie virus A, B (Enterovirus), Crimea-Congo hemorrhagic fever (Bunyavirus), Cytomegalovirus, Cytomegaly, Dengue (Flavivirus), Diptheria (bacteria), Ebola, Ebola/Marburg hemorrhagic fever (Filoviruses), Epstein-Barr Virus ‘EBV’, Echovirus, Enterovirus, Eastern equine encephalitis ‘EEE’, Togaviruses, Encephalitis, Enterovirus, Flavi virus, Hantavirus, Bunyavirus, Hepatitis A., (Enterovirus), Hepatitis B virus (Hepadnavirus), Hepatitis C (Flavivirus), Hepatitis E (Calicivirus), Herpes, Herpes Varicella-Zoster virus, HIV Human Immunodeficiency Virus (Retrovirus), HIV-AIDS (Retrovirus), Human Papilloma Virus ‘HPV’, Cervical cancer (Papovavirus), HSV-1 Herpes Simplex virus Type I, HSV-2 Herpes Simplex virus Type II, HTLV-T-cell leukemia (Retrovirus), Influenza (Orthomyxovirus), Japanese encephalitis (Flavivirus), Kaposi's Sarcoma associated herpes virus KSHV (Herpes HHV 8), Kyusaki, Lassa Virus, Lentivirus, Lymphocytic Choriomeningitis Virus LCMV (Arenavirus), Measles (Rubella), Measles, Measles Micro (Paramyxovirus), Monkey Bites (Herpes strain HHV 7), Mononucleosis (Herpes), Morbilli, Mumps (Paramyxovirus), Newcastle's diseases virus, Norovirus, Norwalk virus (Calicivirus), Orthomyxoviruses (Influenza virus A, B, C), Papillomavirus (warts), Papova (M. S.), Papovavirus (JC—progressive multifocal leukoencephalopathy in HIV) (Papovavirus), Parainfluenza Nonsegmented (Paramyxovirus), Paramyxovirus, ParvoParvovirus (B19 virusaplastic crises in sickle cell disease), Picorna virus, Pertussus (bacteria), Polio (Enterovirus), Poxvirus (Smallpox), Prions, Rabies (Rhabdovirus), Reovirus, Retrovirus, Rhabdovirus (Rabies), Rhinovirus, Roseola (Herpes HHV 6), Rotavirus, Respiratory SyncitialVirus (Paramyxovirus), Rubella (Togaviruses), Bunyavirus, Flavivirus, Poxvirus, Vaccinia virus, Variola, Venezuelan Equine Encephalitis ‘VEE’ (Togaviruses), Wart virus (Papillomavirus), Western Equine Encephalitis “WEE” (Togaviruses), West Nile Virus (Flavivirus), Yellow fever (Flavivirus) and Zika virus (ZIKV).

The virus can be selected from adenovirus, adeno associated virus, alpha virus, flavivirus, herpes simplex virus, lentivirus, Paramyxovirus (i.e. MMR vaccine), coxsackie virus (i.e. CAVATEK), retrovirus, and rhabdo virus.

In an embodiment, the concentration of an amino acids is at least about 0.01%, 0.02%, 0.05%, 0.075%, 0.2%, 0.3%, 0.4%, 0.5%, 0.6%, 0.7%, 0.8%, 0.9%, 1%, 1.5%, 1.75%, 2%, 2.25%, 2.5%, 2.75%, 3%, 3.25%, 3.5%, 3.75%, 4%, 4.25%, 4.5%, 4.75%, 5%, 5.25%, 5.5%, 5.75%, 6%,6.25%, 6.5%, 6.75%, 7%, 7.25%, 7.5%, 7.75%, 8%, 8.25%, 8.5%, 8.75%, 9%, 9.25%, 9.5%, 9.75%, 10%, or more (w/v).

In an embodiment, the concentration of an amino acid is about 0.1% (w/v) to about 10%, or alternatively at about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 10%, 3% to about 10%, 3.25% to about 10%, 3.5% to about 10%, 3.75% to about 10%, 4% to about 10%, 4.25% to about 10%, 4.5% to about 10%, 4.75% to about 10%, 5% to about 10%, 5.25% to about 10%, 5.5% to about 10%, 5.75% to about 10%, 6% to about 10%, 6.25% to about 10%, 6.5% to about 10%, 6.75% to about 10%, 7% to about 10%, 7.25% to about 10%, 7.5% to about 10%, 7.75% to about 10%, 8% to about 10%, 8.25% to about 10%, 8.5% to about 10%, 8.75% to about 10%, 9% to about 10%, 9.25% to about 10%, 9.5% to about 10%, 9.75% to about 10%, 0.1% to about 1%, 0.1% to about 5%, (w/v).

The stability of the virus can be determined by plaque forming unit (PFU), PCR, ELISA, infectivity, and expression of proteins. The virus can be considered stable when the virus in formulation: (a) retains its physical stability and/or (b) retains its biological activity.

In an embodiment, the virus retains its physical stability in a formulation when the virus in formulation: (a) does not precipitate; (b) does not lose infectivity and/or (c) does not lose its capability to express its gene in the host cell.

The physical stability of a virus in a formulation is determined by:(a) visual examination of color and/or clarity; (b) UV light scattering; (c) size exclusion chromatography; and/or (d) electrophoresis.

The biological stability of a virus in a formulation is determined by: (a) infectivity; (b) colony forming unit and/or (c) expression of genes in host cell.

The virus in a formulation can retain a biological activity of between about 50% and about 100%, between about 60% and about 100%, between about 70% and about 100, between about 80% and about 100%, or between about 90% and about 100% as compared to fully live virus stock.

In an embodiment, the virus in a formulation has a biological activity of at least 50%, 55%, 60%, 65%, 70%, 75%, 80%, 85%, 90%, 95% or 100% as compared to a fully live stock of the virus.

In an embodiment, the pH of the pharmaceutical formulation is at least about 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, or 9,

In an embodiment, the pH of the pharmaceutical formulation is from about 3 to about 9, about 4 to about 19, about 5 to about 9, about 6 to about 8, about 6 to about 7, about 6 to about 9, about 5 to about 6, about 5 to about 7, about 5 to about 8, about 4 to about 9, about 4 to about 8, about 4 to about 7, about 4 to about 6, about 4 to about 5, about 3 to about 8, about 3 to about 7, about 3 to about 6, about 3 to about 5, about 3 to about 4, about 7 to about 8, about 7 to about 9, about 7 to about 10.

In an embodiment, the virus concentration is 1, 10, 100, 1,000, 10,000, 100,000, 1,000,000, 1×10⁷, 1×10⁸, 1×10⁹, 1×10¹⁰, 1×10¹¹, 1×10¹², 1×10¹³, 1×10¹⁴, 1×10¹⁵, 1×10¹⁸, 1×10¹⁷, 1×10¹⁸, 1×10¹⁹, or 1×10²⁰ PFU/mL.

In an embodiment, the pharmaceutical formulation comprises two or more different viruses.

In an embodiment, the concentration of each carbohydrate is about 0.01% to about 10%, 0.02% to about 10%, 0.05% to about 10%, 0.075% to about 10%, 0.2% to about 10%, 0.3% to about 10%, 0.4% to about 10%, 0.5% to about 10%, 0.6% to about 10%, 0.7% to about 10%, 0.8% to about 10%, 0.9% to about 10%, 1% to about 10%, 1.5% to about 10%, 1.75% to about 10%, 2% to about 10%, 2.25% to about 10%, 2.5% to about 10%, 2.75% to about 10%, 3% to about 10%, 3.25% to about 10%, 3.5% to about 10%, 3.75% to about 10%, 4% to about 10%, 4.25% to about 10%, 4.5% to about 10%, 4.75% to about 10%, 5% to about 10%, 5.25% to about 10%, 5.5% to about 10%, 5.75% to about 10%, 6% to about 10%, 6.25% to about 10%, 6.5% to about 10%, 6.75% to about 10%, 7% to about 10%, 7.25% to about 10%, 7.5% to about 10%, 7.75% to about 10%, 8% to about 10%, 8.25% to about 10%, 8.5% to about 10%, 8.75% to about 10%, 9% to about 10%, 9.25% to about 10%, 9.5% to about 10%, 9.75% to about 10%, 0.1% to about 10%, 0.1% to about 5%, 0.1% to about 2%, (w/v).

In an embodiment, wherein the concentration of each protein is between about 0.1 mg/ml to about 10 mg/ml, or between about 0.1 mg/ml to about 20 mg/ml, or between about 0.5 mg/ml to about 10 mg/ml, or between about 2 mg/ml to about 20 mg/ml, or between about 3 mg/ml to about 20 mg/ml, such as between 10 mg/ml to about 20 mg/ml, or between 0.5 mg/ml to about 10 mg/ml, or between 1.5 mg/ml to about 10 mg/ml, or between 2 mg/ml to about 10 mg/ml, or between 3 mg/ml to about 8 mg/ml.

In an embodiment, the concentration of each salt is between about 1 mM to about 200 mM, or between about 5 mM to about 150 mM, between about 10 mM to about 100 mM, between about 10 mM to about 25 mM, between about 20 mM to about 150 mM, between about 50 mM to about 150 mM, between about 100 mM to about 200 mM.

In an embodiment, the virus has at least 50%, at least 55%, at least 60%, at least 65%, at least 70%, at least 75%, at least 80%, at least 85%, at least 90% or at least 95% of biological activity relative to fully live virus stock.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A formulation for stabilizing a virus, wherein the formulation comprises:
a. a virus,
b. one or more amino acids at a concentration of about 1-10% w/w,
c. a salt at about 0.01% to 5% w/w,
d. a carbohydrate at about 0.01% to 10% w/w,
e. a protein at about 0.01% to 4% w/w, and
f. water.

2. The formulation for stabilizing a virus of claim 1,
wherein the one or more amino acids are selected from at least three of Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Lysine (K) and Valine (V).

3. The formulation for stabilizing a virus of claim 1,
wherein the one or more amino acids are selected from at least four of Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Lysine (K) and Valine (V).

4. The formulation for stabilizing a virus of claim 1,
wherein the one or more amino acids are selected from at least five of Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Lysine (K) and Valine (V).

5. The formulation for stabilizing a virus of claim 1,
wherein the one or more amino acids are selected from at least six of Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), Lysine (K) and Valine (V).

6. The formulation for stabilizing a virus of claim 1,
wherein each of the one or more amino acids is at a concentration of 0.01% to 0.5%.

7. The formulation for stabilizing a virus of claim 1,
wherein the virus is selected from Adeno-Associated Virus, Adenovirus, Arena virus (Lassa virus), Alpha virus, Astrovirus, Bacille Calmette-Guerin ‘BCG’, BK virus (including associated with kidney transplant patients), Papovavirus, Bunyavirus, Burkett's Lymphoma (Herpes), Calicivirus, California, encephalitis (Bunyavirus), Colorado tick fever (Reovirus), Corona virus, Coronavirus, Coxsackie, Coxsackie virus A, B (Enterovirus), Crimea-Congo hemorrhagic fever (Bunyavirus), Cytomegalovirus, Cytomegaly, Dengue (Flavivirus), Diptheria (bacteria), Ebola, Ebola/Marburg hemorrhagic fever (Filoviruses), Epstein-Barr Virus ‘EBV’, Echovirus, Enterovirus, Eastern equine encephalitis ‘EEE’, Togaviruses, Encephalitis, Enterovirus, Flavi virus, Hantavirus, Bunyavirus, Hepatitis A., (Enterovirus), Hepatitis B virus (Hepadnavirus), Hepatitis C (Flavivirus), Hepatitis E (Calicivirus), Herpes, Herpes Varicella-Zoster virus, HIV Human Immunodeficiency Virus (Retrovirus), HIV-AIDS (Retrovirus), Human Papilloma Virus ‘HPV’, Cervical cancer (Papovavirus), HSV 1 Herpes Simplex I, HSV 2 Herpes Simplex II, HTLV-T-cell leukemia (Retrovirus), Influenza (Orthomyxovirus), Japanese encephalitis (Flavivirus), Kaposi's Sarcoma associated herpes virus KSHV (Herpes HHV 8), Kyusaki, Lassa Virus, Lentivirus, Lymphocytic Choriomeningitis Virus LCMV (Arenavirus), Measles (Rubella), Measels, Measles Micro (Paramyxovirus), Monkey Bites (Herpes strain HHV 7), Mononucleosis (Herpes), Morbilli, Mumps (Paramyxovirus), Newcastle's diseases virus, Norovirus, Norwalk virus (Calicivirus), Orthomyxoviruses (Influenza virus A, B, C), Papillomavirus (warts), Papova (M.S.), Papovavirus (JC-progressive multifocal leukoencephalopathy in HIV) (Papovavirus), Parainfluenza Nonsegmented (Paramyxovirus), Paramyxovirus, Parvovirus (B19 virus aplastic crises in sickle cell disease), Picorna virus, Pertussus (bacteria), Polio (Enterovirus), Poxvirus (Smallpox), Prions, Rabies (Rhabdovirus), Reovirus, Retrovirus, Rhabdovirus (Rabies), Rhinovirus, Roseola (Herpes HHV 6), Rotavirus, Respiratory Syncitial Virus (Paramyxovirus), Rubella (Togaviruses), Bunyavirus, Flavivirus, Poxvirus, Vaccinia virus, Variola, Venezuelan Equine Encephalitis ‘VEE’ (Togaviruses), Wart virus (Papillomavirus), Western Equine Encephalitis “WEE” (Togaviruses), West Nile Virus (Flavivirus), Yellow fever (Flavivirus), and Zika virus (ZIKV).

8. The formulation for stabilizing a virus of claim 1,
wherein the formulation further comprises a surfactant.

9. The formulation for stabilizing a virus of claim 8,
wherein the surfactant is at least one of polysorbate 20 (PS-20), polysorbate 80 (PS-80) or poloxamer 188 (F-68).

10. The formulation for stabilizing a virus of claim 1,
wherein the protein is selected from at least one of albumin, recombinant proteins, cytokines, enzymes, antibodies, plasma proteins, gelatin and soy peptone.

11. The formulation for stabilizing a virus of claim 1,
wherein the protein is present at a concentration of greater than about 10 μg/m L.

12. The formulation for stabilizing a virus of claim 1,
wherein the carbohydrate is selected from at least one of glucose, fructose, lactose, maltose, sucrose, trehalose, sorbitol, mannitol and inositol.

13. The formulation for stabilizing a virus of claim 1,
wherein the carbohydrate is present at a concentration of greater than about 1 mg/mL.

14. The formulation for stabilizing a virus of claim 1,
wherein the salt is selected from at least one of sodium chloride, potassium chloride, magnesium chloride, manganese chloride, sodium phosphate, potassium phosphate, sodium sulfate, potassium sulfate and ammonium sulfate.

15. The formulation for stabilizing a virus of claim 1,
wherein the salt is present at a concentration of greater than about 10 mM.

16. The formulation for stabilizing a virus of claim 1,
wherein the pH of is from about 3 to 9.

17. The formulation for stabilizing a virus of claim 1,
wherein at least one of the one or more amino acids has a negative or positive charged side chain.

18. The formulation for stabilizing a virus of claim 1,
wherein at least one of the one or more amino acids has a hydrophobic or polar uncharged side chain.

19. The formulation for stabilizing a virus of claim 1,
wherein the one or more amino acids is comprised of a first amino acid with a polar uncharged side chain and a second amino acid with a hydrophobic side chain.

20. A method of stabilizing a virus in solution comprising a step of suspending the virus in the formulation of claim 1.

21. A formulation for stabilizing a virus comprising at least three amino acids,
wherein at least one amino acid is selected from the group consisting of glutamic acid (E) and aspartic acid (D), and
wherein at least one amino acid is selected from the group consisting of Alanine (A), Arginine (R), Asparagine (N), Cysteine (C), Glutamine (Q), Glycine (G), Histidine (H), Methionine (M), Proline (P), Serine (S), Threonine (T), Tyrosine (Y), Lysine (K) and Valine (V).

22. A formulation for stabilizing a virus comprising at least three amino acids or amino acid analogs,
wherein at least one amino acid or amino acid analog is selected from the group consisting of Alanine (A), Arginine (R), Asparagine (N), Aspartic Acid (D), Cysteine (C), Glutamic Acid (E), Glutamine (Q), Glycine (G), Histidine (H), Methionine (M), Proline (P), Serine (S), Threonine (T), Tyrosine (Y), and Valine (V), and
wherein at least one amino acid or amino acid analog is selected from the group consisting of Selenocysteine, Citrulline, Cystine, Gama aminobutyric acid (GABA), Ornithine, Theanine, Betaine, Carnitine, Carnosine, Creatine, Hydroxyproline, Hydroxytryptophan, N-acetyl cysteine, S-Adenosyl methionine (SAM-e), Taurine, and Tyramine.
